(19) United States
(12) Patent Application Publication
(10) Pub. No.: US 2006/0147936 A1
(43) Pub. Date: Jul. 6, 2006

(54) Use of a gene mutation in the human gnas gene for predicting risks of diseases, courses of the disease and for predicting the response to disease therapies

(76) Inventors: Ulrich Frey, Essen (DE); Winfried Siffert, Essen (DE)

Correspondence Address: AMSTER, ROTHSTEIN & EBENSTEIN LLP, 90 PARK AVENUE, NEW YORK, NY, 10016, US

(21) Appl. No.: 10/545,998

(22) PCT Filed: Feb. 19, 2004
(86) PCT No.: PCT/EP04/01563

§ 371 Date: Feb. 27, 2006

(30) Foreign Application Priority Data
Feb. 19, 2003 (DE) 103 07 114.8
Oct. 20, 2003 (DE) 103 48 600.3

(51) Int. Cl. C12Q 1/68

(52) U.S. Cl. 435/6

(57) ABSTRACT

The invention concerns the use of a genomic gene modification in the gene for the Gas subunit of the human G protein, coded by the gene GNAS (or GNAS1), for the prediction of disease risks, disease clinical courses and responses to pharmacological and non-pharmacological measures and for the prediction of adverse drug reactions (side-effects). In addition, it concerns the provision of individual gene modifications and haplotypes, with the help of which additional gene modifications applicable for the above purposes can be detected and validated.

The invention concerns the use of a genomic gene modification in the gene for the Gαs subunit of the human G protein, coded by the gene GNAS (or GNAS1), for the prediction of the risks of disease, the course of diseases and the response to diseases therapies by pharmacological and non-pharmacological means and to predict undesired drug effects. In addition, the invention concerns the provision of individual gene modifications and haplotypes, with the help of which additional gene modifications usable for the above mentioned purposes can be detected and validated. Gene modifications of this sort can consist of substitution of thymine by cytosine in position 393 in exon 5, in substitution of guanine by adenine in position -1211, in substitution of thymine by guanine in position -839, or in substitution of thymine by cytosine in position 2291. The gene modifications may be detected singly or in any combination, using procedures familiar to the expert.

Function and Significance of Heterotrimeric G Proteins

All cells in the human body possess membrane receptors on their surface, through which all cell functions are controlled. These receptors include the so-called heptahelical receptors for hormones, neurotranmitters and chemokines. In addition, there are many receptors for growth factors and receptors with intrinsic tyrosine kinase activity, for example, receptors for insulin, insulin-like growth factor, epidermal growth factor, platelet-derived growth factor and many more.

There are in addition many receptors which are responsible for the regulation of hematopoeisis, e.g. the receptor for erythropoietin. For example, cell growth, motility, gene expression, apoptosis and chemotaxis are controlled by receptors of this sort. These receptors transmit their signals into the interior of the cell through the activation of the so-called heterotrimeric G proteins. These G proteins consist of a large family of different α-, β- and γ-subunits. 5 β-Subunits, 13 γ-subunits and more than 20 α-subunits are currently known, which are coded by different genes (Farfel Z et al. The expanding spectrum of G protein diseases. N Engl J Med. 1999 Apr 1;340(13):1012-20).

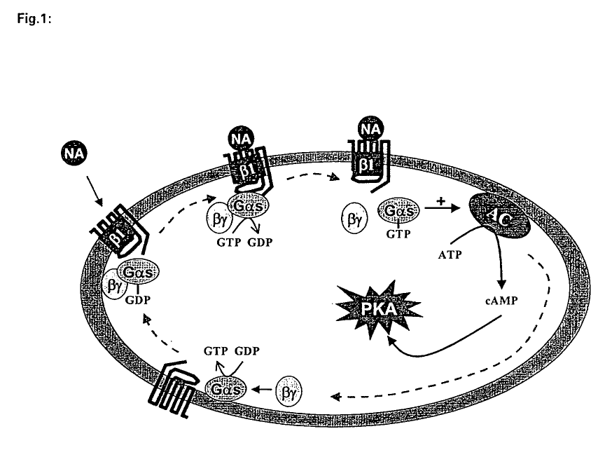

Many different heterotrimeric G proteins are formed by the combination of these different α-, β- and γ-subunits. The isoform combination then determines which heterotrimer can be activated by a defined receptor. The βγ-subunits should be regarded as a functional monomer. In the resting state, the α-subunit has bound GDP (FIG. 1; Legend: NA, noradrenaline; β₁, β₁-adrenergic receptor; Gαs, stimulatory α-subunit, AC, adenyl cyclase; PKA, protein kinase A). After activation of a coupling receptor, the α-subunit releases GDP in exchange for GTP and the βγ-subunits are dissociated from the α-subunits. Both the free α-subunits and the βγ-subunits can direct the activity of a variety of different effectors. These include, for example, ion channels, adenyl cyclase, the PI3-kinase, various MAP-kinases etc. The α-subunits possess intrinsic GTPase activity, which hydrolyses bound GTP to GDP after activation. The βγ-subunits then reassociate with the α-subunit, thus ending the activation cycle. The heterotrimer is then available for a renewed activation cycle (Bourne H R. How receptors talk to trimeric G proteins. Curr Opin Cell Biol. 1997;9(2):134-42). FIG. 1 depicts the G protein cycle. The activation of G proteins of this sort is a decisive step in cell activation. Because of the overwhelming importance of G proteins, it is immediately evident that mutations or genetic polymorphisms in genes which code for G proteins must have a sustained effect on the activability of cells, if these mutations influence the function or expression of G protein subunits. This will then have a decisive effect on the risks of disease or on the course of diseases. In addition, the response to the therapy of diseases, either from drugs or from other measures, such as radiation, diets, operations, invasive treatment etc., depends on the activability of G proteins.

Significance of the Gαs-Subunit

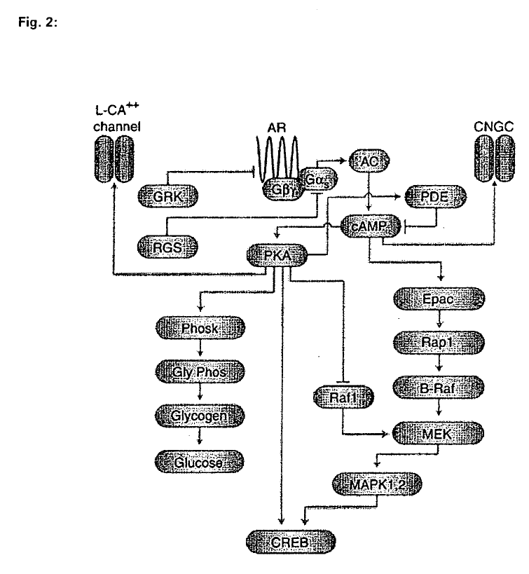

The Gαs-subunit is expressed in all cells of the human body. The effects of its stimulation include the activation of adenyl cyclase, leading to an increase in intracellular cAMP concentration. For example, in this way cAMP-dependent protein kinases can be activated. In addition, other cAMP-dependent signal cascades are inhibited or activated by stimulation of receptors coupled to Gαs. In addition, Gαs can regulate the activity of ion channels, e.g. of potassium or calcium channels (see FIG. 2). The diagram in FIG. 2 shows how the cAMP pathway is coupled to a variety of signal transduction components, including ion channels, transcription factors and metabolic enzymes. AC, adenylyl cyclase; PKA, protein kinase A; PDE, phosphodiesterase; L-Ca⁺⁺ channel, L-type Ca²⁺-channel; CNGC, cyclic nucleotide-gated channel; PhosK, phosphorylase kinase; GlyPhos, glycogen phosphorylase; CREB, cAMP response element-binding protein; EPAC, the cAMP- and AMP-regulated exchange factor for Rap1; Rap1, a small GTPase; MAPK, mitogen-activated protein kinase; Raf1 and B-Raf, MAP kinase kinase kinases; MEK, MAPK/ERK-kinase; MEKK, MAPK/ERK-kinase kinase; GRK, G protein receptor kinase; RGS, “regulators of G protein signaling”; βAR, β-adrenergic receptor (from: Neves at al, G protein pathways. Science. 2002 May 31;296(5573):1636-9). Many receptors couple to Gαs, e.g. the receptors for adenosine, adrenaline, noradrenaline, P₂-purinergic receptors, opioids, dopamine, epidermal growth factor, FSH, VIP, thyroliberin, glucagon, vasopressin, histamine and many more. After stimulation of Gαs-coupled receptors, apoptosis is induced in many cell types, so that there is a connection with tumor diseases and their course and response to therapy and also with inflammatory diseases and their course and response to therapy. In addition, a variety of metabolic pathways are regulated by Gαs. Inhibition of the expression of Gαs in fibroblasts accelerates their differentiation to adipocytes (Wang Hy et al., Antisense oligodeoxynucleotides to GS protein alpha-subunit sequence accelerate differentiation of fibroblasts to adipocytes. Nature. 1992 Jul 23;358(6384):334-7; Wang H et al. G(s)alpha repression of adipogenesis via Syk. J Biol Chem. 1999 Nov 5;274(45):32159-66; review by Neves at al, G protein pathways. Science. 2002 May 31;296(5573):1636-9).

The GNAS Gene

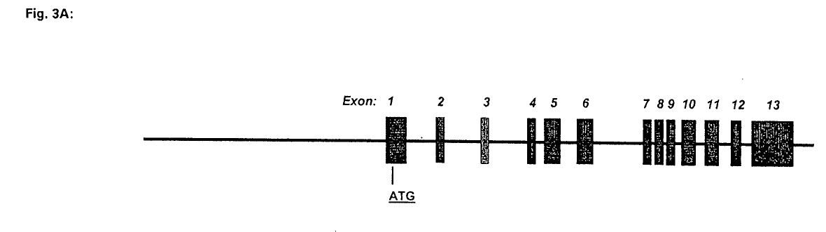

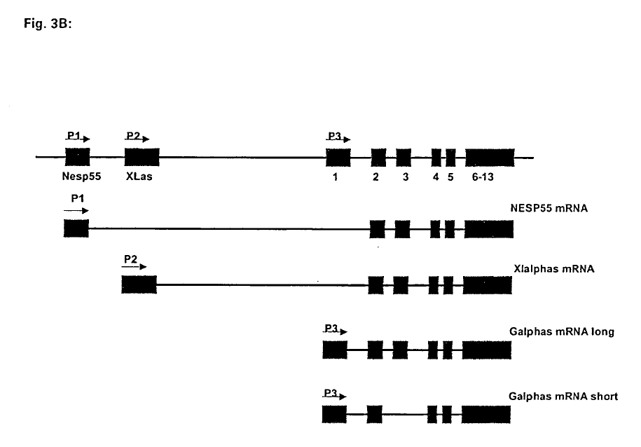

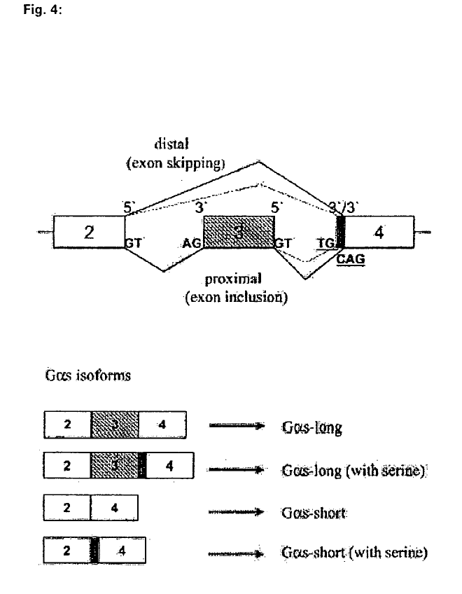

The human Gas gene (GNAS) is localized on chromosome 20q13.2-13.3. It should be pointed out at this point that the terms “GNAS” and “GNAS1” are used synonomously in the literature for this gene. Here we will use the term “GNAS”. FIG. 3A shows a schematic depiction of the intron/exon structure of the human GNAS. The use of different promoters, P1, P2 or P3 leads to the formation of different mRNAs and thus to different gene products(pre-mRNAs), cf. FIG. 3B. Two long (Gαs-1 and -2) and two short (Gαs-3 and -4) isoforms of Gαs result from alternative splicing of exon 3 (T. Kozasa et al. Isolation and characterization of the human Gs alpha gene. Proc. Natl. Acad. Sci. U. S. A 85: 2081-2085, 1988.). The use of an alternative splice-acceptor site for exon 4 leads to insertion of an additional serine in Gₛα-2 and -4 (FIG. 4; P. Bray et al. Human cDNA clones for four species of G alpha s signal transduction protein Proc. Natl. Acad. Sci. U.S.A. 83 (23):8893-8897, 1986). FIG. 4 shows different splice variants of Gαs. The use of different splice sites leads to the inclusion of a CAG triplet (serine) from exon 4. The exclusion of exon 3 leads to the formation of Gαs-short. The splice variants differ with respect to their tissue distribution, their activability by G protein coupled.receptors and their activation by effectors (J. Novotny and P. Svoboda. The long (Gs(alpha)-L) and short (Gs(alpha)-S) variants of the stimulatory guanine nucleotide-binding protein. Do they behave in an identical way? J. Mol. Endocrinol. 20 (2):163-173, 1998; R. Seifert et al. Different effects of Gs alpha splice variants on beta₂-adrenoreceptor-mediated signaling. The Beta₂-adrenoreceptor coupled to the long splice variant of Gs alpha has properties of a constitutively active receptor (J. Biol. Chem. 273 (18):5109-5116, 1998).

Somatic Mutations in GNAS

A series of somatic mutations in the GNAS gene have been described which lead to rare metabolic diseases. These mutations are mostly activating (endocrine GH-secreting tumors, McCune-Albright syndrome, fibrous bone dysplasia) or inactivating (e.g. pseudohypoparathyroidism type IA, pseudopseudohypoparathyroidism, (PPHP)) mutations in the area of the GTP-ase region in the protein. (reviewed in: Weinstein et al., Endocrine Manifestations of Stimulatory G Protein α-Subunit Mutations and the Role of Genomic Imprinting Endocrine Reviews 22 (5): 675-705, 2001). In contrast to single nucleotide polymorphisms (SNPs), these mutations are, for example, not found in the peripheral blood cells of the corresponding patients.

Genomic Mutation in GNAS

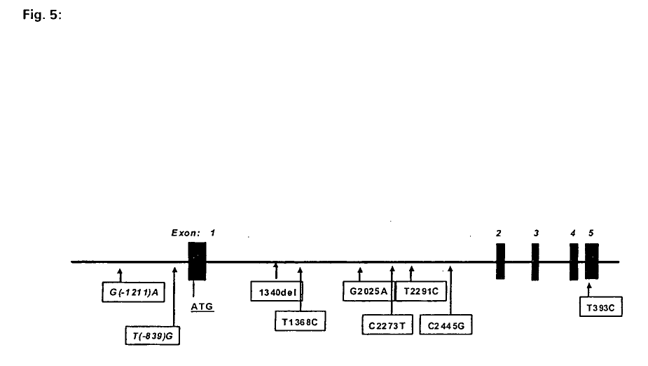

In 1999, Jia et al. investigated whether the GNAS gene contributes to the risk of essential hypertension (Jia H. et al., Association of the Gₛα Gene with Essential Hypertension and Response to β-Blockade Hypertension. 1999;34:8-14.). They investigated a frequently silent T393C polymorphism (SNP, single nucleotide polymorphism) in exon 5 of the GNAS gene (numbering according to the sequence of the cDNA; FIG. 5). This polymorphism is characterized by the lack of a restriction cleavage site for endonuclease FokI (Fok− in the T-allele) or by the presence of this cleavage site (Fok+ in the C-allele). The T393-allele was found more frequently in hypertensives than in normotensive controls. This finding was later confirmed by other investigators (Abe M et al., Association of GNAS gene variant with hypertension depending on smoking status. Hypertension, 2002;40:261-5). In addition, it was reported that hypertensive carriers of the 393C-allele respond better to β-blockers with respect to reduction in blood pressure than do carriers of the T393-allele (Jia H. et al., Association of the Gsα Gene With Essential Hypertension and Response to β-Blockade Hypertension. 1999;34:8-14). Other investigators found a connection between the T393C-status and the risk of orthostatic hypertension (Tabara, Y. et al., Polymorphisms of genes encoding components of the sympathetic nervous system but not the renin±angiotensin system as risk factors for orthostatic hypotension, Journal of Hypertension 2002, 20:651-656). It was thus only shown that the T393C-status was connected with blood pressure regulation and response to β-adrenoceptor blockade. A correlation with other diseases, courses of disease or response to drugs has in general not been described. As this T393C-nucleotide exchange is silent and does not modify the coded aminoacid (isoleucine), there must be other polymorphisms which are in coupling disequilibrium with the T393C polymorphism.

### OBJECT OF THE INVENTION

The invention described here is based on the following goals:
a. To provide function modifying genomic polymorphisms and haplotypes in the GNAS gene which either lead to aminoacid exchange, or
b. influence the splicing behavior, or
c. which lead to modification in protein expression or to modification of the expression of splice variants, or
d. which are suitable for the identification and/or validation of additional polymorphisms or haplotypes in the GNAS gene;
e. To provide nucleotide exchanges and haplotypes which are suited in general for the prediction of disease risks and the course of diseases;
f. To provide nucleotide exchanges and haplotypes which are suited in general for the prediction of the response to drugs and of side-effects;
g. To provide nucleotide exchanges and haplotypes which can in general predict the action of other forms of therapy (radiation; warmth, heat, cold, movement) etc.

Because of the fundamental significance of Gαs for signal transduction, polymorphisms or haplotypes of this sort are suited in general for the prediction of the risks of disease or the courses of disease for all diseases and for the prediction of the response to therapy or failure of therapy or undesired side-effects for all pharmacological or non-pharmacological therapies.

Detection of New Polymorphisms in the GNAS Gene Before Exon 1 and in Intron 1

The main object of the present invention is the identification of the polymorphisms lying before exon 1—G(-1211)A and T(-839)G—and the polymorphisms lying in intron 1—1340del, T1368C, G2025A, C2273T, T2291C and C2445G (FIG. 5), which were found by systematic sequencing of DNA from humans who were homozygotic for the 393C-allele or homozygotic for the T393-allele. For this purpose, gene sequences lying before exon 1 or in intron 1 of GNAS were amplified with the PCR reaction and sequenced according to Sanger's method. The necessary procedure for this, e.g. the derivation of the primer pairs needed for the PCR-reaction and the selection of sequencing primers, are familiar to the expert. New polymorphisms were then found, with either substitution of guanine by adenine in the promotor region in position -1211 (G(-1211)A polymorphism), or with substitution of thymine by guanine in position -839(T(-893)G polymorphism). In intron 1, polymorphisms with the following substitutions were identified: In position 1340, deletion of 12 thymidines, in position 1368 substitution of thymidine by cytosine(T1368), in position 2025, exchange of a 2025 guanine by adenine (G2025A), in position 2273, substitution of thymine by cytosine(C2273T), in position 2291, exchange of thymidine by cytosine (T2291C) and in position 2445, substitution of cytosine by guanine (C2445G). The numbering of these SNPs was performed in such a way that the nucleotide A of the start codon ATG was assigned the number +1. As in accordance with the convention there is no number 0, the nucleotide lying before the A of start codon ATG is assigned the number −1.

The detection of these SNPs in the sense of their use in accordance with the invention can be performed with any procedure familiar to the expert, e.g. direct sequencing, PCR followed by restriction analysis, reverse hybridization, dot-blot or slot-blot procedure, mass spectrometry, Taqman® or light-cycler® technology, Pyrosequencing®. Invader® technology, Luminex procedure etc. In addition, these gene polymorphisms may be simultaneously detected after Mulitplex-PCR and hybridization on a DNA-chip.

Distribution of the T393C, G(-1211)A and T(-839)G Polymorphisms and Derived Genotypes in Different Ethnic Groups, Detection of Haplotypes and Use of These Genotypes in the Identification of Additional Relevant Polymorphisms and Haplotypes

For this purpose, different DNA samples from Caucasians, black Africans and Chinese were genotyped. The results are given in the following table.

GNAS T393C

| | | | |
|---|---|---|---|
| Genotype | Caucasians | Black Africans | Chinese |

| | | | | | | |
|---|---|---|---|---|---|---|
| CC (n, %) | 49 | (25.1) | 6 | (6.0) | 12 | (10.8) |
| CT (n, %) | 97 | (49.7) | 32 | (32.0) | 53 | (47.7) |
| TT (n, %) | 49 | (25.1) | 62 | (62.0) | 46 | (41.4) |
| Total | 195 | | 100 | | 111 | |

| | | | |
|---|---|---|---|
| % T | 50.0% | 78.0% | 65.3% |

This genotype distribution is highly significantly different in the chi² test, with a chi 43.3 and p<0.0001. The T393-allele frequency (%T) is greatest in black Africans.

GNAS G(−1211)A

| | | | |
|---|---|---|---|
| Genotype | Caucasian | Black Africans | Chinese |

| | | | | | | |
|---|---|---|---|---|---|---|
| AA (n, %) | 26 | (13.3) | 0 | (0.0) | 47 | (47.5) |
| GA (n, %) | 104 | (53.3) | 0 | (0.0) | 36 | (36.4) |
| GG (n, %) | 65 | (33.3) | 100 | (100.0) | 16 | (16.2) |
| Total | 195 | | 100 | | 99 | |

| | | | |
|---|---|---|---|
| % G | 60.0% | 100% | 34.3% |

This genotype distribution is highly significantly different in the chi² test, with chi 208.30 and p<0.0001. The (-1211)G-allele frequency (%G) is highest in black Africans, followed by Caucasians and Chinese.

GNAS T(−839)G

| | | | |
|---|---|---|---|
| Genotype | Caucasians | Black Africans | Chinese |

| | | | | | | |
|---|---|---|---|---|---|---|
| GG (n, %) | 10 | (5.1) | 0 | (0.0) | 0 | (0) |
| TG (n, %) | 52 | (26.7) | 1 | (1.0) | 0 | (0) |
| TT (n, %) | 133 | (68.2) | 99 | (99.0) | 100 | (100) |
| Total | 195 | | 100 | | 99 | |

| | | | |
|---|---|---|---|
| % T | 81.5% | 99.5% | 100% |

This genotype distribution is highly significantly different in the chi² test, with chi 72.2 and p<0.0001. The (-839)T-allele frequency (%T) is highest in black Africans and Chinese. It can be deduced from these distributions that, from the evolutionary point of view (relative to Caucasians), the “original states” are the T393-allele, the (-1211)G-allele and the (-893)G-allele. Differences of this sort in genotype distribution in different ethnic groups generally indicate that the associated phenotypes were of importance for evolution and that they brought the carriers of these phenotypes certain advantages. The expert is aware that different genotype distribution in different ethnic groups is an indication that even today certain genotypes or haplotypes are associated with certain diseases or with certain physiological or pathophysiological ways of reacting or responding to therapy, e.g. with drugs.

A further analysis finds a coupling disequilibrium between all three polymorphisms in Caucasians. Coupling disequilibrium means that allele combinations (haplotypes) occur which are clearly statistically more frequent or rarer in combination than would be expected on the basis of their frequency.

The following table shows the distribution of T393C genotypes for Caucasians stratified according to GNAS G(-1211)A genotypes.

| | GNAS G(−1211)A | | | |
|---|---|---|---|---|
| GNAS T393C | AA | GA | GG | Total |
| CC | 2 | 25 | 22 | 49 |
| CT | 11 | 53 | 33 | 97 |
| TT | 13 | 26 | 10 | 49 |
| Total | 26 | 104 | 65 | 195 |

Marked accumulation of the (-1211)G-allele in carriers of the T393-allele is evident (p<0.01 ). The following table shows the distribution of T393C genotypes in Caucasians, stratified according to GNAS T(-893)G-genotypes.

| | GNAS T(−893)G | | | |
|---|---|---|---|---|
| GNAS T393C | GG | TG | TT | Total |
| CC | 5 | 16 | 28 | 49 |
| CT | 4 | 16 | 67 | 97 |
| TT | 1 | 20 | 38 | 49 |
| Total | 10 | 52 | 133 | 195 |

Marked accumulation of the (-893)T-allele in carriers of the T393-allele is evident. The following table shows the distribution of G(-1211)A genotypes in Caucasians, stratified according to GNAS T(-893)G-genotypes.

| | GNAS T(−893)G | | | |
|---|---|---|---|---|
| GNAS G(−1211)A | GG | TG | TT | Total |
| AA | 0 | 0 | 26 | 26 |
| GA | 0 | 32 | 72 | 104 |
| GG | 10 | 20 | 35 | 65 |
| Total | 10 | 52 | 133 | 195 |

Marked accumulation of the (-893)T-allele in carriers of the -1211G-allele is evident (p<0.0001 ).

Additional analysis shows that the haplotypes T393+G(-1211)+T(-839) (60%) and 393C+(-1211)A+(-839)G (47%) occur preferentially in Caucasians. Aside from this, all conceivable permutations are found.

Coupling Disequilibrium of the Intron 1 Polymorphisms to G(-1211)A and T393C

In hundred genotyped Caucasians, the coupling disequilibria of the intron 1 polymorphisms to each other were calculated, together with the coupling disequilibria of the intron 1 polymorphisms to the G(-1211)A and T393C polymorphisms. The counting and assignment of the corresponding nucleotide positions was in such a way that the start codon ATG in exon 1 was assigned to position +1 (see FIG. 5). FIG. 5 shows the structure of GNAS and the positions of new and known polymorphisms. The figure shows the T393C polymorphism in exon 5, together with polymorphisms G(-1211)A and T(-839G) before exon 1. ATG designates the start codon in exon 1. As already mentioned, the numbering of the promoter/enhancer region polymorphisms and of the intron 1 region was performed in such a way that nucleotide A in the start codons ATG in exon 1 was assigned the value +1 and counting then proceeded over the exon-intron border. The nomenclature of the T393C polymorphism in exon 5 corresponds to the numbering in the cDNA. Starting there, the nucleotides were counted continuously over the intron-exon border.

The following table shows the distribution of the C2273T genotypes for Caucasians, stratified according to GNAS A2025G genotypes.

| | | C2273T | | | |
|---|---|---|---|---|---|
| | | CC | CT | TT | Total |
| A2025G | AA | | | 16 | 16 |
| | AG | | 52 | | 52 |
| | GG | 32 | | | 32 |
| Total | | 32 | 52 | 16 | 100 |

The complete coupling of the genotypes of the two polymorphisms is evident. Therefore only the C2273T polymorphism was included in subsequent analysis.

The following table shows the distribution of T1368C and T2291C genotypes in Caucasians, stratified according to GNAS C2445G genotypes.

| | | | T2291C | | | |
|---|---|---|---|---|---|---|
| C2445G | | | CC | CT | TT | Total |
| CC | T1368C | CC | | | 10 | 10 |
| CG | T1368C | TC | | 41 | | 41 |
| GG | T1368C | TT | 49 | | | 49 |
| | Total | | 49 | 41 | 10 | 100 |

Complete coupling of the three polymorphisms is evident. Therefore only the T2291C polymorphism was included in subsequent analysis.

The following table shows the distribution of C2273T genotypes for Caucasians, stratified according to GNAS 1340del genotypes. The “D” stands for the deletion of twelve thymidine residues.

| | | D1340I | | | |
|---|---|---|---|---|---|
| | | DD | ID | II | Total |
| C2273T | CC | | 5 | 27 | 32 |
| | CT | | 49 | | 49 |
| | TT | 16 | | | 16 |
| Total | | 16 | 54 | 27 | 97 |

The almost complete coupling of the genotypes of the two polymorphisms is evident, with a chi squared value of 171.2 (p<0.00001). As there is almost hundred percent coupling with these two polymorphisms too, the C2273T will continue to be considered.

The following table shows the distribution of C2273T genotypes for Caucasians, stratified according to GNAS G(-1211)A genotypes.

| | | G(−1211)A | | | |
|---|---|---|---|---|---|
| | | AA | GA | GG | Total |
| C2273T | CC | | | 32 | 32 |
| | CT | | 52 | | 52 |
| | TT | 16 | | | 16 |
| Total | | 16 | 52 | 32 | 100 |

The complete coupling of the genotypes of the two polymorphisms is evident. Subsequent analysis therefore only considers the G(-1211)A polymorphism.

The following table shows the distribution of G(-1211)A genotypes for Caucasians, stratified according to GNAS T2291C genotypes.

| | | T2291C | | | |
|---|---|---|---|---|---|
| | | CC | TC | TT | Total |
| G(−1211)A | GG | 7 | 15 | 10 | 32 |
| | GA | 26 | 26 | | 52 |
| | AA | 16 | | | 16 |
| Total | | 49 | 41 | 10 | 100 |

The marked accumulation of the 2291T-allele in carriers of the -1211G-allele is evident (chi-squared=42.4, p<0.0001).

The following table shows the distribution of T393C genotypes for Caucasians, stratified according to GNAS T2291C genotypes.

| | | T2291C | | | |
|---|---|---|---|---|---|
| | | CC | TC | TT | Total |
| T393C | CC | 6 | 11 | 7 | 24 |
| | CT | 21 | 25 | 2 | 48 |
| | TT | 22 | 5 | 1 | 28 |
| Total | | 49 | 41 | 10 | 100 |

The accumulation of the 2291C-allele in carriers of the T393-allele is evident(chi-squared≧24.9, p<0.0001).

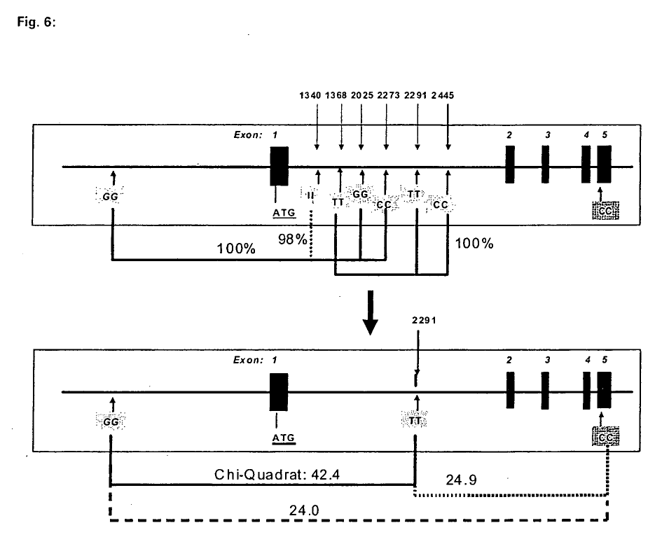

FIG. 6 shows the coupling disequilibria between genotypes of the intron 1 polymorphisms and G(-1211)A and T393C. Polymorphisms which are coupled to =98% are taken together for the sake of simplicity. The quality of the coupling is characterized by the chi-squared value. With a degree of freedom of 3, a value of >7.82 corresponds to significant coupling. The higher this value is, the tighter is the coupling disequilibrium.

Additional analyses shows that the haplotypes T393+(-1211)A+2291C (58%) and 393C+G(-1211)+T2291 (45%) occur preferentially in Caucasians.

Aside from this, all conceivable permutations were found.

One object of this invention is that these new polymorphisms can be used to detect and to validate additional relevant genomic gene modifications in GNAS or in neighboring genes, which, for example, are in coupling disequilibrium with genotypes in the GNAS gene. These can also be genes which are also on chromosome 20, but at greater distances from the GNAS gene. The procedure for this purpose was as follows:
1. For certain phenotypes (cellular properties, disease states, disease courses, response to drugs etc.) an association between the polymorphisms T393C, G(-1211)A, T2291C and T(-839)G and the genotypes in intron 1 is first established, whereby these associations may be established for each genotype separately or by using all permutations of the haplotypes.
2. For newly detected gene modifications in the GNAS or neighboring genes, it is investigated whether existing associations were enhanced or weakened by using the genotypes or haplotypes described above.
   Functional Significance of the T393C Polymorphism and the Polymorphisms Coupled to These

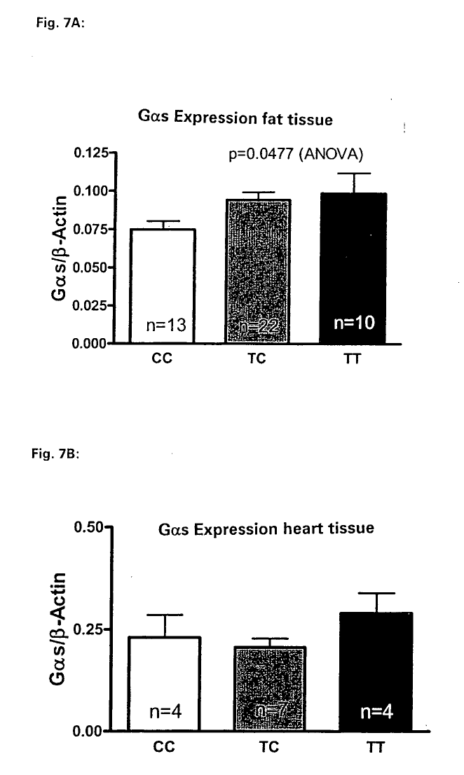

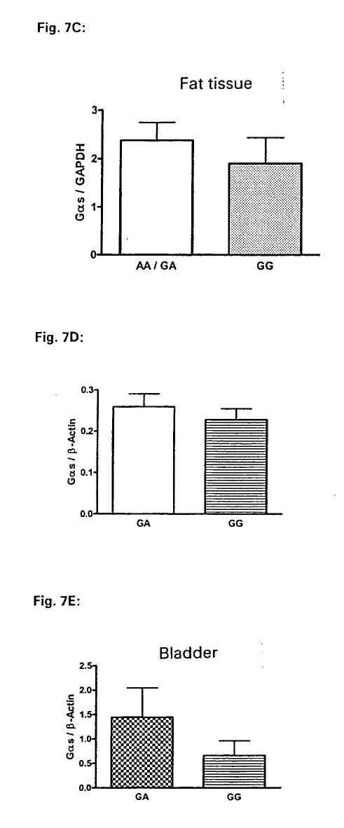

It was investigated which functional modifications can be assigned to gene modifications in the GNAS gene. For example, correlation with alternative splicing, tissue-specific expression or overexpression of the Gαs-protein in dependence on the T393C polymorphism or the A-1211G polymorphism would be conceivable. For this purpose, mRNA was isolated from human heart and fat tissue and transcribed into cDNA using reverse transcriptase. The procedure is familiar to the expert. The level of expression was then determined with real time-PCR (Taqman procedure) and corrected for the level of expression of the housekeeping gene β-actin. The results are shown in FIG. 7. FIG. 7A shows the relative expression of Gαs mRNA (ratio Gαs/β-actin) in human fat tissue, depending on the genotypes of the T393C polymorphism. With T-allele carriers of the T393C polymorphism, there were significantly higher expression values than with homozygotic CC genotypes. Similarly, in human heart tissue there were relatively high expression values for TT genotypes than for the CC- and TC genotypes of the T393C polymorphism (FIG. 7B). FIG. 7B shows the relative expression of Gαs mRNA (ratio Gαs/β-actin) in human fat tissue, depending on the genotypes of T393C polymorphism. Similar considerations apply to the analysis of the A(-1211)G polymorphism. For A-allele carriers, there is in fat tissue (FIG. 7C; shows the relative expression of Gas mRNA (ratio Gαs/GAPDH) in human fat tissue in dependence on the genotypes of the G(-1211)A polymorphism)), in heart tissue (FIG. 7D; shows the relative expression of Gαs mRNA (ratio Gαs/GAPDH) in human heart tissue in dependence on the genotypes of the G(-1211)A polymorphism)) and in urinary bladder tissue (FIG. 7E; shows the relative expression of Gαs mRNA (ratio Gαs/GAPDH) in human urinary bladder tissue in dependence on the genotypes of the G(-1211)A polymorphism))enhanced expression of Gas mRNA, both absolutely and relatively to housekeeping genes (e.g. GAPDH, β-actin).

It has thus been demonstrated that there are genetic modifications in the GNAS gene which modify the expression of Gαs in different tissues. This may be the T393C polymorphism, the A(-1211)G—polymorphism, the T2291C—polymorphism, or polymorphisms, which are in coupling disequilibrium with these. It is thus also a component of the invention described here to quantify the expression of Gαs at the mRNA level or protein level, to associate it with known polymorphisms of the GNAS and to discover and validate new and even more suitable polymorphisms.

These findings of genotype-dependent expression of Gαs in human tissue are of considerable significance. In transgenic animals, the overexpression of Gαs leads to increased apoptosis of cardiac myocytes (Geng Y. et al., Apoptosis of cardiac myocytes in Gs alpha transgenic mice. Circ Res. 1999, 84, 34-42), as a cause of heart failure. There is increased influx of calcium ions into the myocytes of these animals (Kim S J et al., Differential regulation of inotropy and lusitropy in overexpressed Gsalpha myocytes through cAMP and Ca²⁺ channel pathways. J Clin Invest. 1999 Apr;103(7):1089-97). In addition, these animals exhibit increased heart rates and increased blood pressure (Uechi M et al., Depressed heart rate variability and arterial baroreflex in conscious transgenic mice with overexpression of cardiac Gs alpha (Circ Res. 1998 Mar 9;82(4):416-23) The same effects would therefore be expected in humans overexpressing the Gαs protein because of genetic modification of the GNAS. This would be expected to lead to increased cardiovascular risk. This includes increased risks of obesity, hypertension, stroke, coronary heart disease, myocardial infarction, pre-eclampsia etc. Moreover, we would expect differences in the response to substances for which Gαs activates the receptors or to substances which indirectly induce agonists of which the activity is mediated through Gαs. The altered tendency to apoptosis can influence the course of a variety of diseases (e.g. tumor and immune diseases) and determine the response to drugs.

Use of a Gene Modification in GNAS to Predict the Risks of Disease and the Course of Diseases

As the Gαs-subunit has a key role in cell activation, it is an essential component of the invention that in general the risks of diseases and the course of diseases can be predicted by using the gene modifications.

Human heterotrimeric G proteins are composed of the subunits α, β and γ. A series of isoforms of these are known, coded by different genes. For example, there are 13 different γ-isoforms (γ₁-γ₁₃), at least 5 different β-isoforms (β₁-β₅) and many different α-isoforms (αₛ (short and long), αₒ, αᵢ₁₋₃, α_{q}, α_{11-16, α}_{olf} etc.) As G proteins are known to play a central role in controlling the function of all human cells, independently of which cell receptors are activated, it should be expected directly that the course of a variety of totally different diseases would be influenced by a genetically determined, enhanced activability of G proteins. Particularly with the variety of functions of G proteins, mutations modifying function attain extraordinarily great significance and are of great predictive power. This is in contrast to mutations in other genes, e.g. those coding for hormones or hormone receptors.

This means that genetic modifications in proteins which are expressed in all human body cells and which collect all incoming hormonal signals centrally, and which thus regulate cell functions, can decisively influence all physiological and pathophysiological processes or at least modulate them. In addition, responses to drugs will also be decisively influenced. This concerns both desired and undesired drug effects.

It has been repeatedly postulated in the scientific literature that functional modifications in G proteins have a persistent influence on a variety of diseases or on the course of a variety of diseases. These gene modifications may be mutations leading to structural changes in G protein subunits, which may for example modify ability to be activated by a receptor; they may concern the enzymatic GTPase activity or affect the dimerization of the βγ-subunits. In addition, such modifications may alter the composition of heterotrimeric G proteins. Moreover, the level of expression of such G protein subunits may be changed or splice variants with modified function may occur (Farfel Z et al., The expanding spectrum of G protein diseases. N Engl J Med. 1999 Apr 1;340(13):1012-20; Iiri T et al., G protein diseases furnish a model for the turn-on switch. Nature. 1998 Jul 2;394(6688):35-8; Iiri T et al., G proteins propel surprise. Nat Genet. 1998 Jan;18(1):8-10. Spiegel A M. Hormone resistance caused by mutations in G proteins and G protein-coupled receptors. J Pediatr Endocrinol Metab. 1999 Apr;12 Suppl 1:303-9. Spiegel A M. Inborn errors of signal transduction: mutations in G proteins and G protein-coupled receptors as a cause of disease. J Inherit Metab Dis. 1997 Jun;20(2):113-21. Spiegel A M. The molecular basis of disorders caused by defects in G proteins. Horm Res. 1997;47(3):89-96. Spiegel A M. Mutations in G proteins and G protein-coupled receptors in endocrine disease. J Clin Endocrinol Metab. 1996 Jul;81(7):2434-42. Review. Spiegel A M. Defects in G protein-coupled signal transduction in human disease. Annu Rev Physiol. 1996;58:143-70).

The following can be concluded from the above examples:

1. Gene modifications in genes which code for ubiquitously expressed proteins influence a variety of diseases or cause a variety of risks of disease.

2. G proteins control almost all processes in signal transduction in the human body.

3. On the one hand, it is obvious from the cited literature that it is generally assumed that mutations and polymorphisms in the genes coding for G proteins can evoke such diseases. On the other hand, a connection between genomic mutations in the GNAS gene for the GαS-subunit of heterotrimeric G proteins and risks of disease has neither been described nor assumed in the literature.

All diseases which are accompanied by a gene modification in GNAS and which, for example, are determined by a modification in the level of expression of the Gαs protein, can be named as follows:
1. Cardiovascular diseases. These particularly include hypertension, stroke, coronary heart disease and myocardial infarction, heart failure, pre-eclampsia or gestosis. As a connection between the T393C-polymorphimsm and hypertension has already been described (Jia H. et al., Association of the Gₛα Gene with Essential Hypertension and Response to β-Blockade Hypertension. 1999;34:8-14.), it is a component of the present invention that the G(-1211)A-, T2291C- und T(-839)G polymorphisms newly described here and the polymorphisms described as being in complete coupling disequilibrium (intron 1) are also a risk factor for hypertension, as they are in coupling disequilibrium with the T393C polymorphism. As hypertension is generally known to be a main risk factor for stroke, myocardial infarction and heart failure, it is an additional component of the present invention that the gene modifications in GNAS also increase the risk of such diseases.
2. Endeocrinological and metabolic diseases. These include obesity, metabolic syndrome, type 2 diabetes mellitus, gout, osteoporosis, thyroid diseases such as hyperthyroidism and hypothyroidism and Basedow disease, hyper- and hypoparathyroidism, Cushing's disease, hyper- and hypoaldosteronism and many more.
3. Psychiatric diseases, such as depression, schizophrenia, alcoholism and anxiety disorder, phobias, neuroses
4. Neurological diseases such as Parkinson's disease, multiple sclerosis, epilepsy
5. Dermatological diseases, such as psoriasis, neurodermitis
6. Tumor diseases
   Obesity

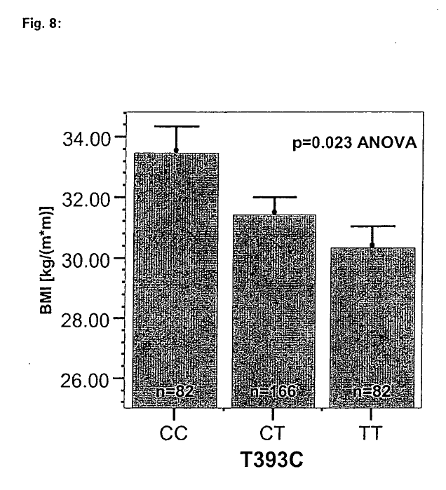

Genetic studies included a genomic scan, to identify genetic markers associated with obesity. Several studies have succeeded in identifying markers associated with obesity on chromosome 20q13 (Lee, J. H. et al. Genome scan for human obesity and linkage to markers in 20q13. Am. J. Hum. Genet. 64, 196-209 (1999). Dong, C. et al. Interacting genetic loci on chromosomes 20 and 10 influence extreme human obesity. Am. J. Hum. Genet. 72, 115-124 (2003)). As the GNAS gene is localized in this region, it could be shown in the context of this invention that the T393C polymorphism is significantly associated with body weight. For this purpose, a meta-analysis was performed of overweight patients with a mean body weight of 90.5 kg (BMI=31.7) Patients were included from three different studies. Patients with the CC genotype exhibited a significantly higher BMI (33.5 kg/m²) than the CT (31.4 kg/m²) and TT genotypes (30.3 kg/m², FIG. 8). FIG. 8 shows the association of the GNAS T393C polymorphism with overweight. The same results are found if the other polymorphisms described here are used.

Obesity is an essential risk factor for hypertension, stroke, cardiovascular diseases, joint damage, gout and many tumor diseases. Use of a gene modification in the GNAS gene to predict the risk of obesity is therefore equally well suited to predict the risks of diseases associated with obesity.

Gene-Dependent Serum Concentration of Leptin and mRNA-Expression in Fat Tissue

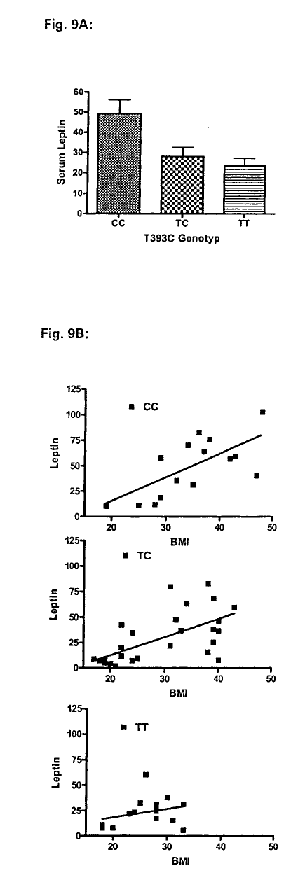

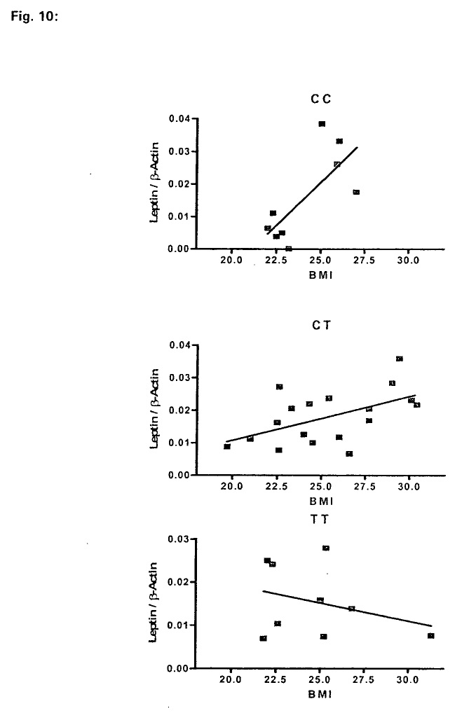

The hormone leptin is predominantly produced in fat tissue and, like all hormones, circulates in the blood circulation of the body. The mode of action of leptin was discovered in experiments in the mouse. This was done by breeding mice without the leptin gene (so-called knock-out mice). These mice exhibited insatiable appetite and became very fat. If these mice were injected with leptin, the eating behavior became normal and they became thin again. Leptin caused a feeling of satiation, increased release of calories, increased fat combustion and decrease in weight. Leptin probably controls these effects through the hypothalamus, the part of the brain where the hunger and thirst center is localized. It has turned out that overweight subjects do not exhibit leptin deficiency, but rather resistance to leptin action. On the other hand, there are many obese subjects with only low serum leptin concentrations. We have investigated whether gene modifications in the GNAS gene determine the serum leptin concentrations (FIG. 9A). FIG. 9A shows the association of the GNAS T393C polymorphism with serum leptin. In CC genotypes, there is a significantly(p<0.005; ANOVA) higher leptin concentration (49.2) than with the CT (28.0) and TT genotypes (23.6). FIG. 9B shows the association of the GNAS T393C polymorphism with serum leptin, in dependence on the Body Mass Index (BMI). Linear regression analysis with CC genotypes found a significantly (p<0.005) greater slope (2.3; p=0.0044) than with the CT (1.78; p=0.0002) and TT genotypes (0.8; p=0.3). With the TT genotypes, there was no significant increase in leptin in dependence on the BMI. To confirm these findings, the expression of leptin-mRNA in human fat tissue was investigated with Real time PCR (Taqman) (FIG. 10). FIG. 10 shows the association of the GNAS T393C polymorphism with the concentration of leptin-mRNA in fat tissue, in dependence on the Body Mass Index (BMI). With CC genotypes, a significantly (p<0.005) greater slope was found in linear regression analysis than with the CT and TT genotypes. With TT genotypes, there was no significant increase in leptin-mRNA in dependence on the BMI.

The expressioin of β-Actin served as control and for the normalization of the values. The procedure is known to the expert. FIG. 10 shows the quotient leptin-mRNA/α-actin-mRNA as a function of the BMI. Taking all the volunteers together, there was a linear correlation between leptin-mRNA expression and BMI (all; slope 0.0012; p=0.04). This correlation is most marked with the CC genotype (slope 0.0049; p=0.0397), or with the presence of the TC genotype (slope 0.0013; p=0.023),but was lacking for the TT genotype (slope—0.00008; p=0.417). Therefore gene modifications in the GNAS gene determine the concentration of leptin serum and the expression of leptin mRNA in fat cells. Genotyping with respect to gene modifications therefore serves to identify patients who would profit from therapy with leptin or with leptin receptor agonists.

Gene-Dependent Apoptosis of Cells

Gene-dependent apoptosis is a strictly regulated physiological process, as a type of “cell suicide”, which plays an important role in the development, maintenance and aging of multicellular organisms and in which individual cells are eliminated according to plan. In accordance with the coupling of apoptosis in a large number of processes which are responsible for the integrity of the whole organism, there are numerous factors which trigger apoptosis. External factors (signals) which trigger apoptosis include ultraviolet, X-rays, gamma rays, oxidation, heat shock (heat-shock response), cytotoxic drugs (cytotoxins)and heavy metals. Many factors (of external or internal nature) which trigger oxidative stress are also apoptosis signals, so that oxygen radicals (free radicals) are of decisive importance in the induction of apoptosis. The involvement of apoptosis has been recognized in numerous diseases. Heart diseases (e.g. myocardial infarction), neurodegenerative diseases (Parkinson's disease), AIDS—destruction of the T-cells of the immune system, osteoporosis, degenerative arthritis (rheumatism) are examples. On the other hand, inhibition of apoptosis can lead to cancer. Many types of cancer are characterized by the overexpression of genes which inhibit apoptosis (e.g. bc1-2) or mutations in the corresponding genes (e.g. p53; p53 protein). Some substances used in tumor therapy induce apoptosis in sensitive tumor cells. Cells of the immune system (immune cells) are also subject to apoptosis while they mature; T-lymphocytes which recognize a self-antigen (autoantigen)are removed in the thymus (thymocyte development) by apoptosis. This means protection from autoreactive cells (autoreactivity). If autoreactive immune cells are not eliminated by apoptosis, this can lead to autoimmune diseases. If there are genetic factors which generally modify the ability of the cells to undergo apoptosis, this is coupled to a general change in the risk of disease and the course of a variety of diseases in which apoptosis is important is modified. This applies to cardiovascular diseases (myocardial infarction, coronary heart disease, heart failure, stroke), inflammatory diseases (rheumatism, joint diseases, Bechterew's disease, psoriasis, neurodermitis), tumor diseases, HIV infection, viral liver infections (hepatitis B and C), rejection reactions after organ transplantation and many more.

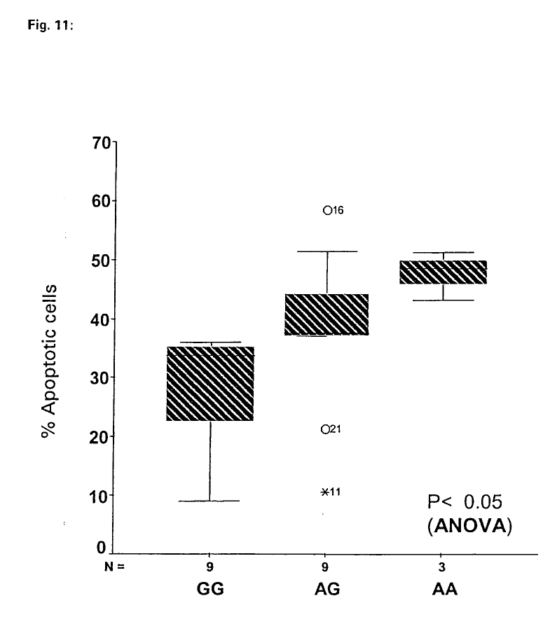

We show here that gene modification in the GNAS gene is a suitable method of determining the spontaneous apoptosis behavior of cells. For this purpose, the apoptosis of B-cells in patients with chronic lymphatic leukemia was investigated. Apoptosis was quantified after 24 h incubation of the cells in the medium by FACS analysis with annexin V/PI dye. Procedures for the quantification of apoptosis are known to the expert. The results are summarized in FIG. 11.

FIG. 11 shows that the gene modifications in the GNAS gene (G(-1211)A polymorphism) determine the spontaneous apoptosis of B-cells in patients with chronic lymphatic leukemia. The lowest number of apoptotic cells is found in subjects with the GG genotype. The cells were genotyped with respect to GNAS G(-1211)A polymorphisms. The statistically significantly enhancement of apoptosis is evident in cells from patients with the AA and AG genotypes and, as already mentioned, relatively low apoptosis in patients with the GG genotype is also found. This behavior is transferable to all other cells of the human body which express GaS.

Fundamental Properties of Malignant Tumors

In malignant tumors, also known as cancer, there are characteristic changes in fundamental functions, which support the undesired growth of cells of this sort. Cancer cells are characterized by a loss of contact inhibition and uncontrolled cell growth. Changes of this sort are triggered by many toxins, so-called carcinogens, which damage hereditary material. These toxins include many chemicals, tobacco smoke and UV light. In addition, genetic factors play a dominant role in the origin of cancer. Cancer cells are characterized not only by their uninhibited growth, but also by their tendency to produce daughter growths (metastases) in other organs. Metastases are regularly spread by the circulation of the blood or through lymph vessels. In many cases, cancer is not curable and leads to death. Therapeutic attempts are made to remove the initial tumor and the metastases by operation. In addition, tumors can be irradiated. Using so-called cytostatics, antibodies to certain proteins or cell surface markers or immune modulators (cytokines, interferons), it is attempted to kill the rapidly dividing cancer cells or to move them into programmed cell death (apoptosis). Currently available therapeutic measures only prolong life in most cases, but do not lead to a definite cure.

Prognostic Factors in Cancer

Definition of prognostic factors for the clinical course of cancer is of considerable medical significance. These should provide information on the response to certain forms of therapy or be generally predictive for the occurrence of metastases, tumor progression and survival. Prognostic factors generally known to the medical expert have been used. For example, these include the size of the tumor, its depth of penetration into the surrounding tissue, growth beyond organs, penetration into blood or lymph vessels or lymph nodes or the degree of differentiation of the tumor cells. In addition, there are some relatively unspecific serological markers. The procedure for classifying tumors is generally referred to as “staging” and “grading”. It is generally the case that the present of distant metastases and a low degree of differentiation are very unfavorable prognostic factors. It is nevertheless the general experience in medicine that patients with the same tumor stage can exhibit drastically different clinical courses. In some patients, there is rapid progression of the disease and metastases and relapses occur, in other patients the disease stops developing for unclear reasons. Metastases then can be local, regional or distant from the initial tumor. For this it is necessary that a large number of malignant cells are washed through the lymphatic or blood circulation into neighboring tissue, or passed on by direct contact. A tendency to relapse means the recurrence of a tumor after incomplete or partial operative removal of the tumor. This is not renewed malignant transformation, but regrowth of tumor tissue which had not been fully removed. Relapse from metastases is also possible, as these can remain latent for many years. The term progression means the recurrence of a tumor with higher grading (more dedifferentiation) or the reappearance of metastases.

There are very evidently many individual unrecognized biological variables which are major determinants of the clinical course of a tumor disease, unrelated to staging or grading. These factors include genetic host factors.

It is then desirable to develop genetic markers which are predictive for the occurrence of tumors. These markers fulfill the function of assuring that affected individuals are given additional screening measures at an early stage (serology, X-ray, ultrasound, NMR etc.). In this way, cancer can be recognized at an early stage and therapy attempted, thus greatly increasing the chances of cure and survival, as these are much better in early stage than in advanced tumors.

Types of Tumors

In general, all cells in the human body can be malignantly transformed and lead to cancer. The mechanisms of tumor progression, metastasis and therapeutic progression described above and later are generally applicable. These mechanisms and claims therefore apply to all human tumors, in particular to the following tumors:

Tumors of the urogenital tract: in particular, carcinoma of the urinary bladder, prostate carcinoma, renal cell carcinoma and seminoma.

Tumors of the female genital organs: Mammary carcinoma, uterus carcinoma, ovarian carcinoma, cervical carcinoma.

Tumors of the gastrointestinal tract: carcinoma of the oral cavity, esophageal carcinoma, gastric carcinoma, liver carcinoma, bile duct carcinoma, pancreatic carcinoma, colon carcinoma, rectal carcinoma.

Tumors of the respiratory tract: larynx carcinoma, bronchial carcinoma.

Tumors of the skin: malignant melanoma, basalioma, T-cell lymphoma

Tumors of the hematopoeitic system: Hodgkin's and non-Hodgkin's lymphoma, acute and chronic leukemia etc.

Tumors of the brain or nervous tissue: glioblastoma, neuroblastoma, medulloblastoma, meningeal sarcoma, astrocytoma.

Soft tissue tumors, for example, sarcomas and tumors of the head and neck

G Proteins and Malignant Cell Transformations

in vitro experimental studies show that mutations in G proteins subunits can cause malignant transformations in cells. The expression of a constitutively active Gαq-subunit lacking GTPase-activity leads to malignant transformation of fibroblasts (Kalinec G et al., Mutated alpha subunit of the Gq protein induces malignant transformation in NIH 3T3 cells. Mol Cell Biol. 1992 Oct;12(10):4687-93). Similar observations have been made for the α-subunits Gα12, Gα13, Go, Gz and Gαi2(Xu N et al., A mutant alpha subunit of G12 potentiates the eicosanoid pathway and is highly oncogenic in NIH 3T3 cells. Proc Natl Acad Sci U S A. 1993 Jul 15;90(14):6741-5. Xu N et al Potent transforming activity of the G13 alpha subunit defines a novel family of oncogenes.

Biochem Biophys Res Commun. 1994 Jun 15;201(2):603-9. Pace A M et al A mutant alpha subunit of Gi2 induces neoplastic transformation of Rat-1 cells. Proc Natl Acad Sci U S A. 1991 Aug 15;88(16):7031-5. Wong Y H et al., Mutant alpha subunit of Gz transforms Swiss 3T3 cells. Oncogene. 1995 May 18;10(10):1927-33). These studies demonstrate that mutations in the α-subunits can in principle contribute to malignant cell transformation.

G Protein Mutations and Cancer in Man

Somatic mutations in the subunits Gαs and Gαi2 have been detected in some rare adenomas in man. These are designated as Gip2 (Gαi2-subunit)or as Gsp (Gαs-subunit). These are not genetic host factors which modulate the course of the disease, but causal factors (reviewed by: Farfel Z et al. The expanding spectrum of G protein diseases. N Engl J Med. 1999;340(13):1012-20).

Use of Gene Modifications in the GNAS Gene to Predict the Course of Tumor Diseases

An important component of the present invention is the provision of diagnostically relevant gene modifications in the GNAS gene as a prognostic factor for all human tumor diseases. In the nature of the matter, all tumor diseases cannot be described. Therefore, the principle will be described in selected examples, which demonstrate its general applicability:

### EXAMPLE 1

Chronic Lymphatic Leukemia (CLL)

Chronic lymphatic leukemia is a chronic form of leukemia. This disease is characterized by a large number of degenerated lymphocytes. A total of 30% of all leukemia cases are chronic lymphatic leukemia. The mean age at which this disease first occurs is 65 years. Only ten percent of the patients are under 50 years old. Men are about two to three times more frequently affected than are women. No risk factors are known for the development of CLL. The disease is nevertheless rare in Japan and China. Even Japanese immigrants into the USA fall extremely rarely ill from CLL. This fact indicates that genetic factors play a role. The therapy depends on the stage of the disease. CLL can show a benign course for up to 20 years. This means that the patient exhibits no symptoms apart from enlarged lymph nodes and possibly tiredness and loss of appetite. Treatment only starts when the number of lymphocytes starts to rise above a certain extent, the proportion of red blood cells and the number of blood platelets sinks or other complications occur. Early treatment has no effect on the course or the result of the disease. Chemotherapy is the most important therapeutic measure. In certain cases, the patient must also be irradiated or operated on. Patients can live up to 20 years with the diagnosis of CLL, without exhibiting severe symptoms. However, the more advanced the disease is, the greater is the damage to health because of modifications to organ systems. The doctor can assess the prognosis of the disease on the basis of the Binet stage. The characteristics of the stage of CLL include the number of lymphocytes in the blood and bone marrow, the size of the spleen and liver and whether the patient is anemic or not. CLL leads to changes in the immune system, so that patients suffering from CLL are more at risk of developing other types of cancer. However, the clinical course of patients with the same Binet stage can be quite disparate. It is a component of this invention to show that gene modifications in the GNAS gene are suited to predict the clinical course of CLL. For this purpose, patients with CLL were genotyped with respect to the described genetic modifications in GNAS and the gene status compared with the progression of the disease. Progression is defined here as the time interval between the first diagnosis of CLL and the necessity for therapy.

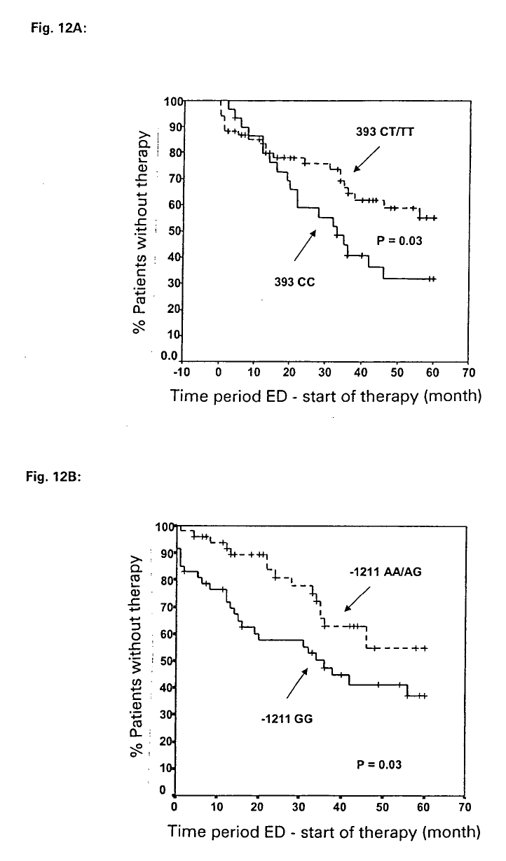

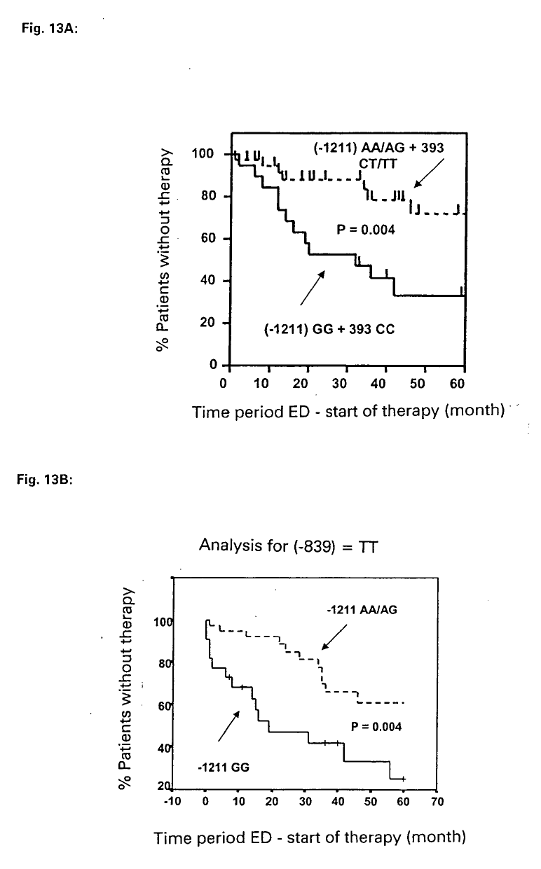

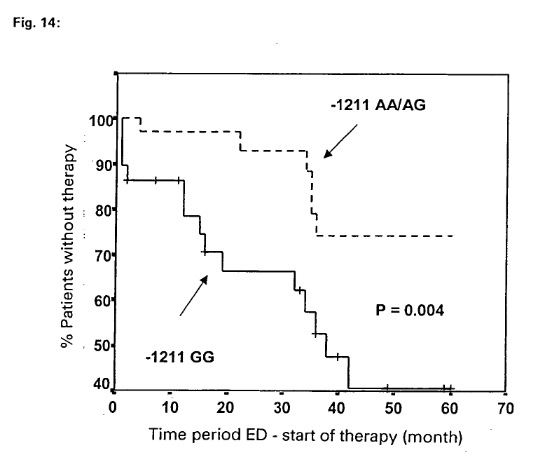

FIG. 12A shows the GNAS T393C status and the progression of the disease in patients with CLL (ED=first diagnosis). It is evident that there are significant differences with respect to freedom from therapy, depending on the GNAS T393C status and whereby the CC genotype is associated with an acceleration of twofold in the disease progression (Hazard Ratio 2.07; p=0.02). FIG. 12B shows the GNAS G(-1211)A status and disease progression in patients with CLL (ED=first diagnosis). It is evident that there is a significant difference with respect to freedom from therapy, depending on the GNAS G(-1211)A status, whereby the GG genotype is associated with an acceleration of three fold in disease progression (Hazard Ratio 3.01; p=0.01). The effect is particularly marked in the combined analysis of the alleles or genotypes (-1211) GG+393 CC versus (-1211)AG/AA+393 CT/TT (FIG. 13A). In a similar manner, analyses of the G(-1211)A status and the T(-839)G status can be combined, as is shown in FIG. 13A. The combination with the T2291C status is also sensible, as this polymorphism is in coupling disequilibrium both with the G(-1211)A- and with the T393C polymorphism. FIG. 14 shows that gene modifications in GNAS can be used for the prognosis of CLL, particularly in the early stage (BINET A), as is shown for the G(-1211)A status.

### EXAMPLE 2

Urinary Bladder Carcinoma

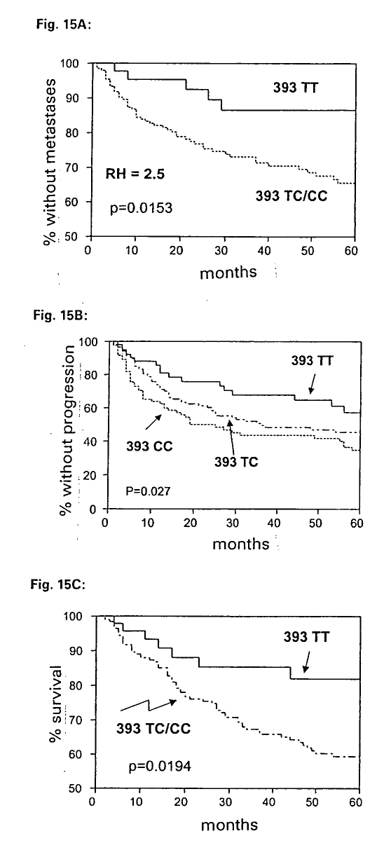

Bladder carcinoma is a malignant tumor in the mucous membrane of the bladder. Bladder cancer occurs most often in patients aged between 60 and 70. Men are three times more often affected than women. Bladder cancer is the third most frequent form of cancer in men, after lung cancer and prostate cancer. Bladder cancer can be caused by external influences. The risk factors include smoking, continuous stress to the organism from chemicals, such as dyes or analgesic abuse. Investigation of many patients shows that the tumor is superficial. This can be removed operatively with the help of a cystoscope. More than 70% of the patients treated for a superficial bladder carcinoma develop a recurrent tumor later. More than half of the recurrent tumors are non-muscle invasive. These can be treated or controlled by transurethral resection. It is therefore important to recognize the lesions early and to monitor the patient regularly and closely. The most important control is cystoscopy with urine cytology. Regular elimination urograms serve to control possible manifestations of the tumor in the renal pelvis and ureters. There has hardly been any valid marker which is predictive for the subsequent course of the disease. Classical factors such as depth of penetration, degree of differentiation, metastasis, lymph node involvement, etc., are therefore currently used for prognosis. Genetic markers for tumor progression, tendency for relapses, probability of survival and response to therapy would bring a major improvement in the care of patients with bladder carcinoma. A further component of the invention is that the use of the gene modification in GNAS is suitable for the prediction of the further clinical course of the disease. FIG. 15A shows the time point up to the occurrence of metastases, depending on the GNAS T393C status. The risk of metastasis in patients with the TC/CC genotype is increased by a factor of 2.5 (95%: 1.2-4.0; p=0.015). The median time till metastasis is 29 months with the TT genotype, but 13 months with the combined TC/CC genotype. A similar relationship is found if the time up to tumor progression is examined (FIG. 15B). Progression is understood as the occurrence of metastases or the recurrence of the tumor with higher staging or grading. The shape of the curve is significantly different for the 393 TT-, TC- and CC-genotypes (p=0.027, Log rank test), where the CC- and TC genotypes are assigned to the less favorable clinical course. Finally, the connection between the GNAS T393C status and survival is depicted (FIG. 15C). Here too it is evident that patients with the GNAS 393 genotypes TC and CC die earlier than patients with the TT genotype. The median time of survival is 44 months with the TT genotype, but only 20.5 months for the combined TC/CC genotype. Because of the significant coupling disequilibrium to the T393C polymorphism, the G(-1211)A- and the T2291C- polymorphisms can be used in a similar manner. It can be assumed that the increased tendency to apoptosis caused by increased Gαs expression in T393 carriers and -1211A- or 2291C carriers can have a favorable effect on the course of the disease.

### EXAMPLE 3

Renal Cell Carcinoma

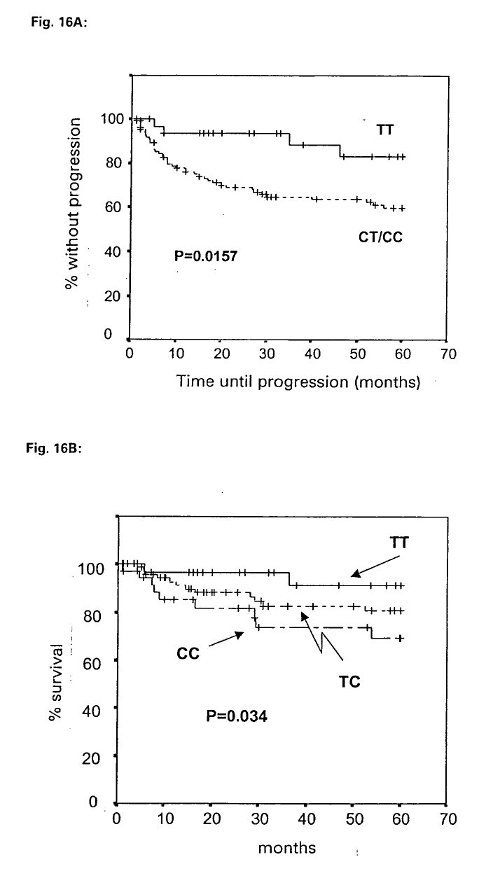

Kidney cancer is a serious disease. The chances of cure depend on the size and extension of the tumor. For patients without metastases, the 10-year survival rate is up to 80%, but with considerable intra-individual variability. As a consequence of the currently wide use of ultrasound techniques, many of the tumors are already recognized at an early stage, before metastasis, and can therefore be treated in time. If distant metastases are present, the operative removal of one kidney may be combined with subsequent immune therapy with interleukin-2 or interferon-alpha. This increases the endogenous defenses of the body against cancer. For metastatic renal cell carcinoma, the combination interferon, interleukin-2 and 5-fluorouracil has given a response rate of 36 percent in studies. There is currently no predictive marker for progression, survival or response to therapy for patients with renal cell carcinoma. By using the GNAS T393C polymorphism, it was possible to recognize accelerated progression in patients with the TC/CC genotypes (FIG. 16 A). In addition, it is shown here that the survival of patients with renal cell carcinoma depends on the GNAS-T393C status (FIG. 16B). Thus, the patients with the CC genotype die earliest, followed by the TC- and TT genotypes, where the last live longest. In the multivariate analysis, GNAS status (p=0.012), grading (p<0.001) and stage (p<0.001) are independent predictors for survival. Because of the significant coupling disequilibrium to the T393C polymorphism, the G(-1211)A polymorphism can be used in a similar manner.

### EXAMPLE 4

Acute Myeloid Leukemia (AML)

The cells of most importance for AML are the granulocytes and the monocytes. The main function of these cells is immune defense. The underlying cause of AML is degeneration of the stem cells. This degeneration has the effect that the stem cell loses its ability to differentiate into functioning blood cells, although their tendency to multiply is fully maintained. There is then malignant multiplication of immature cells, the blasts. These proliferate too rapidly and without adequate control in the bone marrow and are released too soon into the blood. They therefore do not attain adequate maturity and thus are unable to carry out their duties, such as immune defense. As the room in the bone marrow is limited, the leukemic cells displace developing red blood cells and platelets. Too few erythrocytes and platelets are produced. The consequences are anemia and excessive tendency to bleeding. As the leukemia cells circulate through the blood and lymphatic vessel systems, they migrate to the lymph nodes, spleen and liver too and impair their function. These organs often increase in size. Acute myeloid leukemia should therefore be seen as a malignant expansion and loss of ability to differentiate of the hematopoeitic cells in the bone marrow. This predominantly affects cells of the myeloid lineage—monocytes and granulocytes. Chemotherapy is a fundamental component of the treatment of patients with acute myeloid leukemia. In this way, it is attempted to inhibit the growth of leukemia cells by adding drugs. Drugs of this sort are also called cytostatics. Chemotherapy is complemented by two other therapeutic options: stem cell transplantation and bone marrow transplantation.

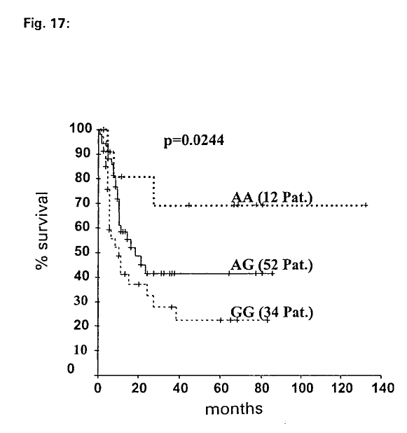

It has unfortunately not yet been possible to develop drugs which selectively damage leukemia cells. The side-effects of chemotherapy are caused by damage to healthy cells which naturally divide rapidly. Cytostatics—drugs which disturb cell division—are mostly administered in combination and according to a scheme based on fixed time intervals (regimen). In ca. 70 to 80% of patients, complete remission—regression of all signs of the disease—is achieved after 2 to 3 treatment cycles. Intensive chemotherapy destroys 99 to 99.9% of all leukemia cells. However the remaining leukemia cells in most patients cannot be destroyed, in spite of intensive consolidation therapy. After 5 years, only about 20 to 40% of the patients are still alive. It was then investigated whether gene modifications in the GNAS gene can serve as prognosis factors for the survival of patients with AML. A significant correlation was found between the genotype status relative to the A(-1211)G-polymorphism. FIG. 17 shows that patients with acute myeloid leukemia with the AA genotype of the A(-1211)G-polymorphism have the best chance of survival, while the course of the disease is accelerated with the AG- and GG genotypes. All patients included in the study had received intensive chemotherapy and/or bone marrow transplantation. This then demonstrates not only that gene modifications in the GNAS gene are markers for the progression of AML, but that these gene modifications can also be used as general pharmacogenetic markers for cancer chemotherapy and for bone marrow transplantation.

Possible Molecular Causes for GNAS-Dependent Tumor Progression.

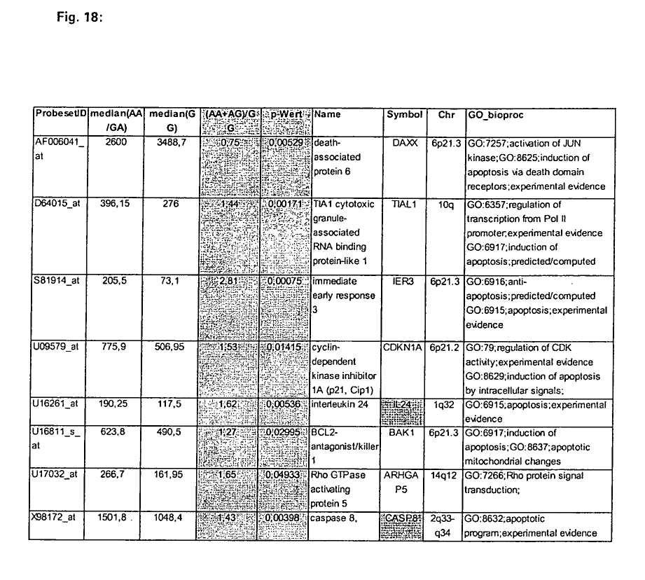

For this purpose, cDNA from CLL cells was hybridized with Affymetrix gene chips, as described in the literature (Dürig J et al., Expression of ribosomal and translation-associated genes is correlated with a favorable clinical course in chronic lymphocytic leukemia. Blood. 2003 Apr 1;110(7):2748-55). The level of expression of the gene was investigated, not according to the clinical course, but according to the status of the GNAS G(-1211)A polymorphism. The result is shown in FIG. 18. FIG. 18 shows gene expression in the lymphocytes of patients with CLL, in dependence on the GNAS G(-1211)A genotype. As already mentioned, the cDNA was hybridized on gene chips from Affymetrix and the intensity of the hybridization signal was quantified. The expert is familiar with the procedure for quantitative gene analysis. Statistically significant greater expression of apoptosis-related genes is evident in AA/AG genotypes in comparison with the GG genotype. The findings with all 30,000 genes on the Affymetrix chip can of course not be discussed. Interestingly, there is significantly increased expression in (-1211)A-allele carriers in comparison to the GG genotype of apoptosis-associated genes, for example of caspase 8, immediate early response 3, IL24, CDKN1a etc It is therefore an obvious assumption that enhanced signal transduction through Gαs—as a consequence of increased expression—may contribute to increased tendency to apoptosis. This shows that gene modifications in the GNAS gene can be used to discover genes relevant to the genes and new sites of attack for drugs.

Use of Gene Modifications in the GNAS Gene to Predict Risks of Disease and Clinical Courses

As the wide variety of functions of Gαs is well known, gene modifications in the GNAS gene can increase the risk for many different diseases or influence the clinical courses. It is not possible in principle to investigate all human diseases and their clinical courses. However, we have taken four different diseases as examples: obesity, CLL, AML, bladder carcinoma and renal cell carcinoma. These data prove unambiguously that gene modifications in the GNAS gene can be used for the purpose described here. There is no a priori connection between these diseases.

Pharmacogenetics—Diagnosis of the Efficacy of Drugs, Their Potency and the Occurrence of Adverse Effects

Principles and Goals of Pharmacogenetics

The efficacy of drugs and/or the occurrence of side-effects are defined by a series of parameters, aside from the specific chemical properties of the chemically defined products. Two important parameters, the maximal plasma concentration and the plasma half-life, determine the efficacy or lack of efficacy or the occurrence of side-effects to a large extent. Factors determining the plasma half-life include the rate of metabolism of drugs in the liver or other body organs to active or inactive metabolites and the speed of elimination from the body, which can be through the kidneys, through respiratory air, through sweat, through seminal fluid, through stools or through other body secretions. The efficacy after oral administration is limited by the so-called “first-pass effect”, as, after absorption of a drug from the intestine, a defined proportion is metabolized in the liver to inactive metabolites.

Mutations or polymorphisms in the genes of metabolizing enzymes can modify their activity by modifying their aminoacid composition in such a way that the affinity to the substrate to be metabolized can be increased or decreased, so that metabolism is accelerated or slowed down. In a similar manner, mutations or polymorphisms in transport proteins can modify their aminoacid composition in such a way that the transport and thus elimination from the body is accelerated or slowed down.

In the selection of the optimally suited substance for a patient, the optimal dosage, the optimal formulation and for the avoidance of undesired side-effects—which can damage health or be fatal—the knowledge of genetic polymorphisms or of mutations which lead to modification of the gene products is extraordinarily important.

The Action of Hormones in the Human Body and the Significance of Polymorphisms in Hormone Receptors

Many hormones and peptide hormones of the human body and receptor antagonists exert their activity by acting on the so-called receptors in body cells. These are proteins of various structures. After activation of these receptors, the signals must be transmitted into the interior of the cell, which is mediated by activation of heterotrimeric G proteins. Proteins of this type are composed of different α-, β- and γ-subunits. These receptors can be classified into certain groups, depending on their activability by defined hormones. The expert is aware that mutations or polymorphisms in certain receptors can determine the efficacy of certain agonists or antagonists on these receptors. Thus a frequent Gly16Arg-polymorphism in the gene that codes for the β₂-adrenoceptor influences the strength of response to the β₂-sympathomimetic drug salbutamol (Martinez F D, et al. Association between genetic polymorphisms of the beta₂-adrenoceptor and response to albuterol in children with and without a history of wheezing. J Clin Invest. 1997 Dec 15;100(12):3184-8). Polymorphisms in the D₂-receptor gene determine the frequency of the occurrence of dyskinesia in the treatment of Parkinson's disease (Oliveri R L, et al.; Dopamine D₂ receptor gene polymorphism and the risk of levodopa-induced dyskinesias in PD. Neurology. 1999 Oct 22;53(7):1425-30). Polymorphisms in the u-opiate receptor gene determine the analgesic efficacy of opiates (Uhl G R, et al. The mu opiate receptor as a candidate gene for pain: polymorphisms, variations in expression, nociception, and opiate responses. Proc Natl Acad Sci U S A. 1999 Jul 6;96(14):7752-5).

These gene modifications in specific receptors can only be used in the diagnosis of the actions of drugs to the extent that these drugs are specific agonists or antagonists of the receptors under consideration. On the other hand, it would be desirable to have individual diagnosis of the general response to all drugs and the individual prediction of the risk of adverse effects under therapy with drugs.

Diagnosis of the Activability of G Proteins Permits General Diagnosis of the Efficacy of Drugs, Their Optimal Dosage and the Occurrence of Side-Effects

The expression “drug” generally means substances which are added to the human body from the outside, in order to produce defined states. These substances can be hormones, low or high molecular weight substances, peptides or proteins, antibodies or many others.

Most of the drugs used to treat diseases, physical malfunction or impairment in well-being, are hormones, agonists on hormone receptors or other substances which directly or indirectly influence the expression of receptors or the concentration of hormones. A series of drugs exert their influence in that, during therapy with such substances, physiological counter-regulation takes place which raises the concentration of hormones which activate the G protein-coupled receptors. A generally known example which may be mentioned is treatment with diuretics, in particular, loop diuretics and thiazide diuretics. The loss of sodium chloride occurring during therapy leads to the activation of the renin-angiotensin-aldosterone systems. The increased levels of the hormone angiotensin II formed stimulate increased absorption of sodium in the kidney, stimulate salt uptake, increase blood pressure through a direct vasoconstrictory effect on the cells of vascular smooth muscle and induce proliferation processes. It is generally known that these mechanisms evoked by angiotensin II occur after coupling of the hormone to receptors which mediate their activity through activation of heterotrimeric G proteins. The efficiency of these actions is predictable if the strength of the activability of G proteins can be diagnosed. Other drugs exert their activity by inhibiting the re-uptake of transmitters released from neurones, e.g. noradrenaline, adrenaline, serotonin or dopamine. As an example, the drug sibutramine may be given, which inhibits the re-uptake of serotonin and noradrenaline in the central nervous system, as a consequence of which the feeling of hunger is reduced and thermogenesis is increased. Corresponding to this, sibutramine can be used for the therapy of obesity. As noradrenaline and serotonin activate G protein-coupled receptors, the diagnosis of the activability of G proteins is particularly well suited for the prediction of the efficacy of sibutramine and the occurrence of typical, sibutramine-associated side-effects (e.g. increase in heart rate and blood pressure).

The invention is based on the fact that a procedure was invented which is generally suitable for the diagnosis of the activability of G proteins. For this purpose, one or several polymorphisms in the GNAS gene are investigated that code for the human Gαs-subunit of heterotrimeric G proteins. Those polymorphisms are particularly suited which predict the diagnosis or the non-occurrence of an alternative splice procedure of the gene or modified expression of Gαq. With overexpression, there is predictably increased activability of hetrotrimeric G proteins and increased activability of all cells of the human body. Thus determination of the presence of polymorphisms in GNAS permits the diagnosis of the efficacy and adverse effects of drugs, in particular, agonists and antagonists of all receptors of which the activity is mediated through heterotrimeric G proteins. In addition, those polymorphisms in GNAS can be used to diagnose the actions of drugs which, either indirectly or as a consequence of counterregulatory mechanisms of the body, raise or lower the concentrations of endogenous hormones, of which the receptors activate hetrotrimeric proteins. Thus the invention allows the diagnosis of actions and side-effects of all drugs and is not limited to drugs which influence specific receptors in an agonistic or antagonistic manner. In addition, the diagnosis of the allelic or haplotype status in GNAS can be used to determine the individual optimal and tolerated dosage of drugs.

For the diagnosis or increased or reduced activability of G proteins, detection of the TT393C polymorphism or of the G(-1211)A-polymorphism or of the T(-839)G polymorphism or of the T2291C polymorphism are suitable, either alone or in all possible combinations.

In addition, all other gene modifications in GNAS can be used for diagnosis which are in coupling disequilibrium to these polymorphisms and/or also increase or decrease the alternative splice process or the expression.

These gene modifications can be detected with any procedure known to the expert, e.g. direct sequencing, restriction analysis, reverse hybridization, dot-blot or slot-blot procedure, mass spectrometry, Taqman or light cycler technology, pyrosequencing etc. In addition, these gene polymorphisms can be simultaneously detected on a DNA chip after Mulitplex-PCR and hybridization. In addition, for the diagnosis of increased activability of G proteins, other procedures can be used which permit the direct detection of the level of expression of Gαs or of the splice variants of Gαs.

The described procedure is particularly suitable for the diagnosis of the activity of agonists or antagonists on receptors the activity of which is known to be mediated by G proteins. The following examples are named for this purpose and this list of examples could be extended:

1. Adrenergic receptors, in particular α- and β-adrenoceptors and their isoforms and subgroups, i.e. α₁- and α₂-adrenoceptors and β₁-, β₂-, β₃- and β₄-adrenoceptors

2. Muscarinic receptors and their isoforms, e.g. m₁-, m₂-, m₃-, m₄- and m₅-muscarinic receptors and their subtypes. Typical antagonists on muscarinic receptors are, for example, atropine, scopolamine, ipratroprium, pirenzepine and N-butylscopolamine. Typical agonists are carbachol, bethanechol, pilocarpine etc.

3. Dopamine receptors, e.g. D₁-, D₂-, D₃-, D₄-, and D₅-receptors and their isoforms and splice variants

4. Serotonin receptors, e.g. 5-HT₁- 5-HT₂-, 5-HT₃-, 5-HT₄-, 5HT₅-, 5HT₆- und 5-HT₇-receptors and their subtypes, isoforms and splice variants. Typical agonists are sumatriptan and cisapride; antagonists are for example ondansetrone, methysergide, buspirone and urapidil.

5. Endothelin receptors and their subtypes, isoforms and splice variants

6. Bradykinin receptors, e.g. B₁- and B₂-receptors and their subtypes, isoforms and splice variants

7. Angiotensin receptors, e.g. AT II type 1 and type 2 receptors; typical antagonists on the AT II-receptor are losartan and other sartans.

8. Receptors for endorphins and opiates, e.g. the μ-opiate receptor

9. Chemokine receptors CCR1-12 and CXCR1-8 for e.g. interleukin-1/2/3/4/5/6/7/8/9/10/11/12, RANTES, MIP-1α, MIP-1β, stromal cell-derived factor, MCP1-5, TARC, lymphotactin, fractalkine, eotaxin 1-2, NAP-2, LIX etc.

10. Adenosine receptors and their subtypes, isoforms and splice variants

11. Receptors for thrombin (protease-activated receptors)

12. Receptors for lyso-phophatidic acid, phosphatidic acid; receptors for sphingosine phosphate and their derivatives

13. Receptors for prostaglandins and thromboxanes, e.g. for PGE1, PGE2, PGF, PGD2, PGI2, PGF2α, thromboxane A2, etc. 14. Receptors for neuropeptides, e.g. NPY1-5

15. Histamine receptors, e.g. H₁- and H₃-receptors

16. Receptors for platelet-activating factor (PAF-receptor)

17. Receptors for leukotrienes

18. Receptors for insulin, glucagon, insulin-like growth factor (IGF-1 and IGF-2), epidermal growth factor (EGF) and platelet-derived growth factor (PDGF)

19. Receptors for growth hormone (GH), somatostatin (SSTR1-5), thyreotropic hormone (TSH), oxytocin, prolactin, gonadotropins

20. Receptors for cytokines, e.g. interferons

21. Receptors for purines

22. Orphan receptors, of which the activity is mediated by G proteins

23. Receptors for leptin

24. CpG-Oligonucleotide

Moreover, the activity can be predicted of drugs which influence the re-uptake, metabolism or de novo synthesis of neurotransmitters or, during therapy with which, there are changes in the expression or sensitivity of the above named receptors (e.g. sibutramine, fluoxetine). In addition, the actions of all drugs can be diagnosed which directly, indirectly or as the consequence of a physiological counter-regulation, change the concentrations of agonists which activate the above receptors. The effect of radiation therapy on cancer patients can also be predicted.

In particular, the actions and side-effects of the following drugs from the following areas of indication can be diagnosed:

1. Antihypertensive drugs, e.g. β-blockers (propanolol, bisprolol, etc.), diuretics (hydrochlorothiazide and other thiazide diuretics; furosemide, piretanide and other loop diuretics, chlorthalidone), α₁-adrenoceptorblockers (e.g. doxazosin, prazosin), angiotensin receptor blockers (e.g. losartan), ACE-inhibitors(enalapril, captopril, ramipril etc.), Ca²⁺-channel blockers (e.g. nifedipine, verapamil, amlodipine, felodipine), clonidine, reserpine, _ennin-inhibitors

2. Drugs for the treatment of heart failure, e.g. β-blockers (e.g. propanolol, metoprolol), ACE-inhibitors (e.g. captopril, enalapril, ramipril, etc.), angiotensin receptor blockers (e.g. losartan), digitalis glycosides, catecholamines, diuretics.

3. Drugs for the treatment of low blood pressure or heart failure, e.g. α- and β-sympathomimetics (effortil, adrenaline, noradrenaline, dobutamine, β-adrenoceptor blockers, ACE-inhibitors, angiotensin II-receptor blockers.)

4. Drugs for the treatment of migraine, e.g. sumatriptan, rizatriptan, zolmitriptan and other agonists for serotonin receptors, β-blockers (propanolol, timolol), ergotamine and dihydroergotamine

5. Analgesics of the morphine type(morphine, codeine, etc.)

6. Drugs for the treatment of coronary heart disease, such as adenosine, β-blockers (e.g. propanolol, acebutolol), nitrates and Ca²⁺-channel blockers

7. Drugs for the treatment of psychiatric diseases (schizophrenia, manic-depressive diseases, psychoses, depression) and addictive disease, such as alcoholism, (e.g. fluoxetine, paroxetine, imipramine, desipramine, doxepin, mianserin, trazodone, lofepramine), anxiety syndrome (diazepam, etc.), which, for example, influence the dopaminergic, serotoninergic or adrenergic systems. Also drugs which act through receptors for GABA, glycine or glutamate or their derivatives.

8. Drugs for the treatment of Alzheimer's disease (e.g. tacrine) and for the treatment of Parkinson's disease (e.g. bromocriptine, L-DOPA, carbidopa, biperidene, seleginil, etc.) which influence transmitter concentrations of, e.g. muscarinergic or dopaminergic substances.

9. Drugs for the treatment of bronchial asthma, which, for example, either possess direct bronchodilatory activity or antiinflammatory activity, e.g. salbutamol, terbutaline, albuterol, theophylline, montelukast, zafirlukast, cromoglycic acid, ipratropium bromide. This group of drugs also includes antibodies to certain proteins and receptors.

10. Drugs for the treatment of disturbances in the motility of the stomach or intestine and drugs for the treatment of irritable bowel syndrome, e.g. N-butylscopolamine, pirenzepine, metoclopramide)

11. Drugs for the treatment of obesity, which either directly activate receptors with lipolytic activity, e.g. β₃-adrenergic agonist, or are centrally active, e.g. sibutramine, or similar substances which alter satiety or which influence thermogenesis. This also includes drugs which influence gastric emptying.

12. Drugs for the treatment of chronic inflammatory processes or disturbances in the immune system, e.g. cytokines (interferons) in the therapy of virus hepatitis or interleukin-2 in HIV infection. These diseases also include Crohn's disease, ulcerative colitis, asthma, psoriasis, neurodermitis, hay fever. This also includes antibodies to cytokines or to cytokine receptors, e.g. to TNFα

13. Drugs for the treatment of gestosis, pre-eclampsia/eclampsia and the HELPP syndrome.

14. Drugs for the treatment of disturbances in fertility or to rectify menstrual abnormalities in the women or for contraception.

15. Drugs for the treatment of cardiac arrythmias

16. Antidiabetic drugs (acarbose, insulin, troglitazone, metformin, etc.)

17. Hypnotics, antiemetics and antiepileptic drugs

18. Drugs for the treatment of disturbances in sexual function, e.g. erectile dysfunction, female sexual dysfunction, lack of libido, disturbances in orgasm (phosphodiesterase inhibitors such as sildenafil, prostaglandin E1, agonists to dopamine receptors, e.g. apomorphine, yohimbine, phentolamine)

19. Drugs for the treatment of cancer and chemotherapeutic drugs, e.g. 5-fluoruracil, antibodies to proteins and receptors (e.g. to HER-2), substances which block tyrosine kinases etc.

20. Drugs for the treatment of allergic and tumor diseases, in which the effect is achieved by administration of CpG-nucleotides.

21. Drugs for the treatment of obesity, the metabolic syndrome or diabetes, e.g. sibutramine, orlistat, leptin, topiramate, glinide, glitazone, biguanide etc.

22. Drugs for the treatment of the HIV-infection, including antibodies and receptor blockers. Prediction of the occurrence of lipodystrophy during treatment with proteinase inhibitors.

It is of course not possible in the context of the invention described here to prove that all drug actions are determined by the GNAS gene status. In the nature of things, it is also impossible to investigate the genotype-dependent actions of drugs which will only be developed and used in future. On the contrary, examples of drugs are presented with a range of different mechanisms of actions, so that these findings can be generalized.

With the first drug (sibutramine), the effect is triggered through blocking the re-uptake of serotonin and noradrenaline in the central nervous system. In this manner, the concentration of this transmitter is increased extracellularly and in the synaptic cleft. These transmitters then stimulate G protein coupled receptors. The substance sibutramine itself does not stimulate these receptors.

Metformin is taken as the second example. This is an oral antidiabetic drug which has long been known; the molecular-mechanism of action has not been exactly defined. Nothing is known of direct involvement of G proteins.

The third example we discuss is the action of isoprenaline, which is known to activate G protein-coupled receptors directly.

We thus illustrate our thesis of the general predictability of drug action with a substance which activate G proteins directly(isoprenaline), a substance which raises the concentration of other substances which then activate G proteins and a substance which has no effect on G proteins.

### EXAMPLE 1

Efficacy of Sibutramine

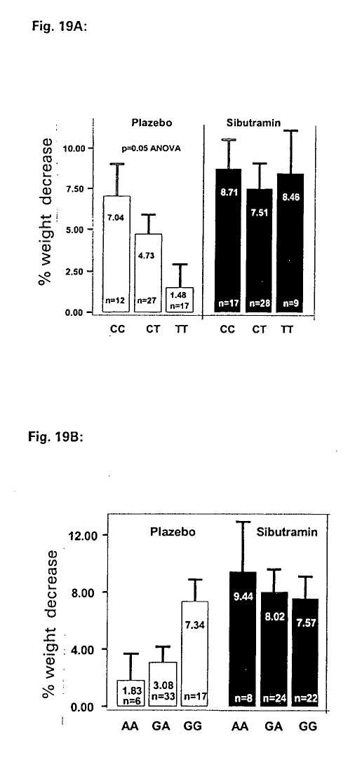

In the context of a placebo-controlled, double blind study, obese patients were given 15 mg sibutramine/day for one year. Sibutramine is a centrally active re-uptake inhibitor of noradrenaline and serotonin, both of which act through G protein-coupled receptors. Sibutramine (trade-name: Reductil, Meridia) enhances the feeling of satiety and makes it easier to lose weight in the context of structured measures to reduce weight (increase in physical activity, reduction in calorie intake). The outcome parameter of the study was the weight loss after one year. The genotype-dependent comparison of placebo vs. sibutramine is shown in FIG. 19A.

FIG. 19A shows the dependence of the action of sibutramine against placebo, in the context of the therapy of obesity. The placebo or sibutramine (15 mg/day) were administered for one year. The loss of weight after one year (%) is illustrated, in dependence on the GNAS T393C genotype.

In the placebo group, patients with GNAS 393 CC- or CT genotype could quite clearly lose weight better(7.0 and 4.7%, respectively) than those with the TT genotype (1.5%). The administration of sibutramine only leads to a clear (p=0.027) increase in the loss of weight in carriers of the TC- and TT genotype, whereas this favorable drug effect was not observed in patients with the CC genotype. Thus therapy with sibutramine is predominantly indicated for patients with TT/TC genotype, whereas patients with CC genotype can also lose weight well under placebo.

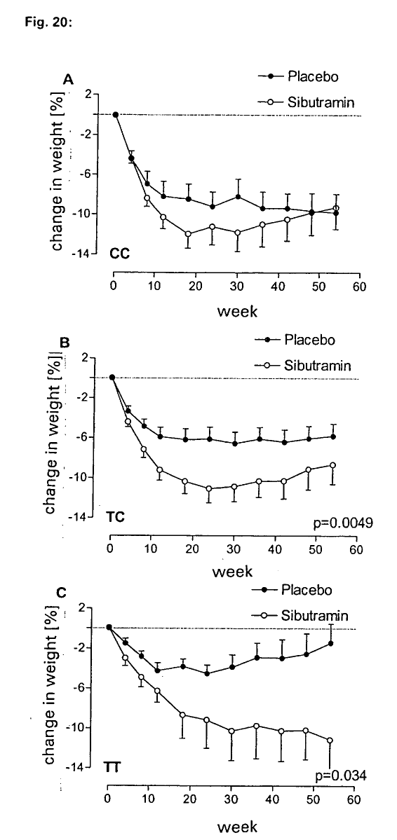

In addition, the study population was genotyped with respect to the GNAS G(-1211)A polymorphism (FIG. 19B). FIG. 19B shows the dependence of the action of sibutramine vs. placebo in the context of the treatment of obesity. Placebo or sibutramine (15 mg/day) was administered for one year. The loss of weight (%) after one year is illustrated in dependence on the GNAS G(1211)A genotype. In the placebo group, patients with GNAS (-1211) GG genotype clearly could lose weight better(7.3%) than those with the AA- and AG genotype (1.8 and 3.1%, respectively). Administration of sibutramine leads to a marked (p=0.027) increase in the loss of weight, but only in carriers of the AG- and AA genotype, whereas this favorable drug effect was not observed in patients with the GG genotype. Thus therapy with sibutramine is predominantly indicated for patients with AG/AA genotype, whereas patients with CC genotype can also lose weight well under placebo. The time courses of the loss of weight for each genotype of the 393CT393C polymorphism (sibutramine versus placebo) are shown in FIGS. 20A, 20B and 20C. FIGS. 20 A, B and C illustrate the individual time courses for the changes in weight with placebo or sibutramine for the GNAS 393 CC genotype (A), the TC genotype (B) and the TT genotype (C). It is evident that only the TC and TT genotypes benefit from therapy with 15 mg sibutramine daily, whereas CC genotypes can lose weight without drug.

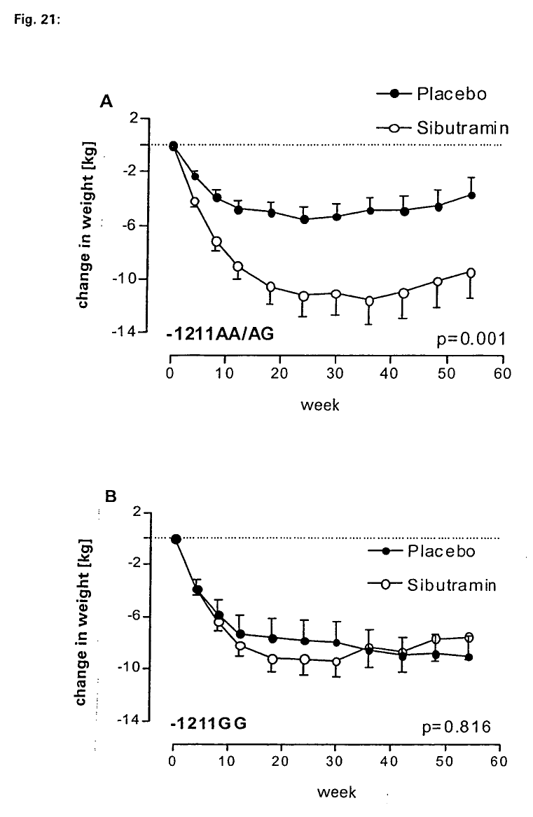

The time courses for the weight loss for each genotype of the G(-1211)A polymorphism (sibutramine versus placebo) are illustrated in FIGS. 21A and 21B. FIGS. 21A and B show the individual time courses for the weight changes with placebo or sibutramine for the GNAS -1211 AA/AG genotype (A) and the GG genotype (B). It is evident that only the AA/AG genotypes benefit from therapy with 15 mg sibutramine daily, whereas GG genotypes can lose weight without drug.

This then also demonstrates that the prediction of the success of non-pharmacological measures for weight reduction is possible by using gene modification in the GNAS gene. This includes structured weight loss programs (e.g. Optifast, Weight Watchers, other training programs), taking satiating food sources (CM3, BMI23) or low calorie foods.

### EXAMPLE 2

Efficacy of Oral Antidiabetics for Weight Loss and for Improving Insulin Sensitivity

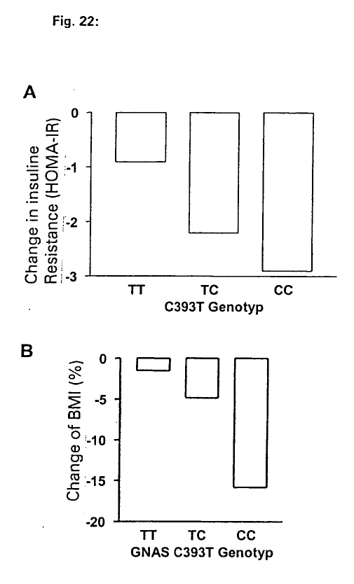

Polycystic ovarian syndrome (PCOS) is a frequent endocrine disease, characterized by chronic anovulation and hyperandrogenism. About 5% of all premenopausal women are affected by this. Most women with PCOS exhibit insulin resistance coupled to an increased risk of the metabolic syndrome (obesity, type 2 diabetes, disturbances in lipid metabolism and hypertension). Treatment of PCOS patients with metformin improves fertility and androgen levels, reduces insulin resistance and facilitates weight reduction. For this purpose, women with the PCOS syndrome were treated with metformin for 12 months. Changes in the Body Mass Index (BMI) and insulin resistance according to the HOMA-IR method (homeostasis model assessment for insulin resistance) were investigated, together with the GNAS genotypes of the T393C polymorphism. FIG. 22A shoes the influence of therapy with an oral antidiabetic(metformin) on insulin resistance(A) and Body Mass Index (BMI; B) in women with polycystic ovaries. As already mentioned, therapy was carried out with metformin for 12 months. The change in the HOMA-IR index is given as a measure of the insulin resistance. It is evident that the most marked improvement of the HOMA-IR index is in individuals with the CC genotype(2.9), in contrast to 2.2 with the TC genotype and only 0.9 with the TT genotype. Thus individuals with the CC and TC genotypes exhibit the most marked improvements in insulin sensitivity under metformin therapy, whereas those with the TT genotype only benefit slightly. The changes in the BMI (a measure of body fat) behave similarly. The decrease here is 15.8% with the CC genotype, whereas the decrease in the BMI with the TC and TT genotypes was only 4.8 and 1.5%, respectively (FIG. 22B). This therefore shows that gene modifications in the GNAS gene can be used to predict the efficacy of oral antidiabetics. This includes not only metformin, but also the so-called glitazones, tolbutamide, sulfonylureas and their derivatives, glinides and arcabose.

### EXAMPLE 3

Prediction of the Action of Catecholamines

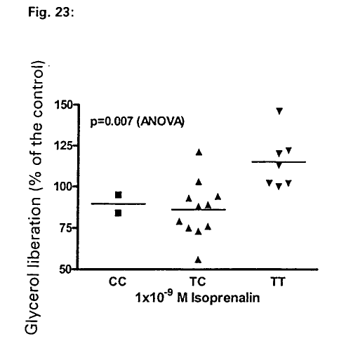

For this purpose, human fat cells ex vivo were stimulated with isoprenaline and lipolysis quantified on the basis of glycerol release (Hauner H et al. Effects of the G protein beta3 subunit 825T allele on adipogenesis and lipolysis in cultured human preadipocytes and adipocytes. Horm Metab Res. 2002;34(9):475-80). The most marked stimulation of lipolysis was found in fat cells from individuals with the GNAS 393 TT genotype (FIG. 23). This agrees with the observation of increased expression of Gαs protein in this genotype.

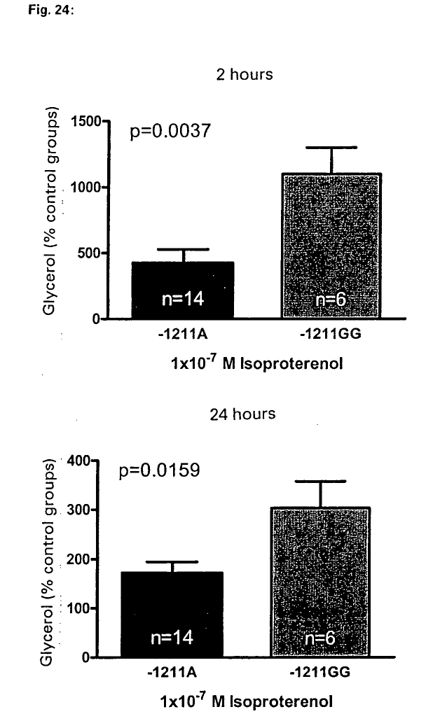

For the G(-1211)A polymorphism, there was the most marked stimulation of lipolysis with the GG genotype (FIG. 24). FIG. 24 exhibits the isoprenaline-stimulated lipolysis in human fat cells in dependence on the GNAS G(-1211)A status. In fat cells from humans with the GG genotype, there is increased lipolysis, measured as the release of glycerol. For this polymorphism, no correlation with changes in expression was evident, suggesting that there is another mechanism than with the T393C polymorphism. This also agrees with the observation that persons with the GG genotype on a low calorie diet lose more weight than persons with the AA/AG genotype.

It has thus been shown that gene modifications in the GNAS gene can be used to predict the action of catecholamines. This is then relevant to substances which directly stimulate α- and/or β-adrenoceptors and also substances which induce increased release of catecholamines. In addition, side-effects can be predicted, when administration of different substances causes activation of the sympathetic nervous system (sildenafil and similar PDE inhibitors).

### EXAMPLE 4

Prediction of Cardiovascular Side-Effects Under Therapy With Sibutramine

Because of the mechanism of action (central inhibition of the re-uptake of noradrenaline and serotonin), during treatment with sibutramine typical side-effects develop such as dry mouth, insomnia and constipation. However, the cardiovascular side-effects are more dangerous, e.g. increases in heart rate and blood pressure, which can lead to tachycardia and myocardial infarction. It has not been possible to predict which patients will develop this.

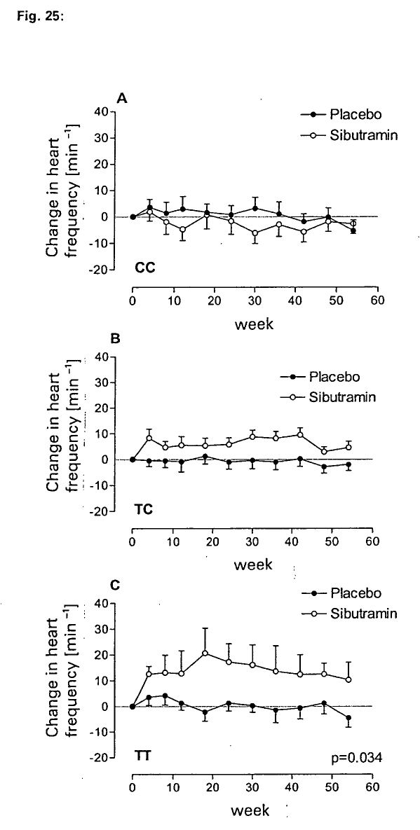

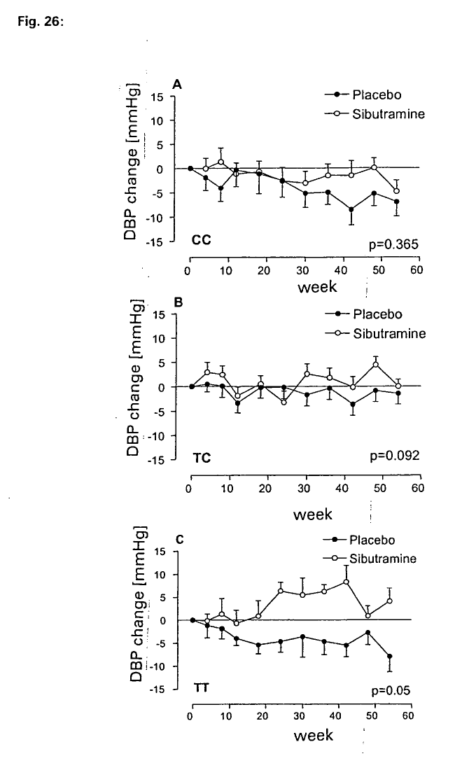

The connection between gene modifications in the GNAS gene and the change in heart rate was examined after treatment with sibutramine versus placebo (FIG. 25). The effects are illustrated as they depend on the T393C polymorphism. It is evident that, with the T393C (FIG. 25B) and TT genotypes (FIG. 25C), there is a significant increased heart rate with sibutramine (versus placebo), by 10 (TC) to 20 beats per minute. This effect does not occur with CC genotypes (cf. FIG. 25A). FIG. 26 makes clear the possible use of a gene modification in the human GNAS gene to predict the increase in diastolic blood pressure during treatment with sibutramine. The illustration shows the time dependent changes in diastolic blood pressure (DBP), in dependence on the genotype of the T393C polymorphism. As shown in FIG. 26, during therapy with sibutramine, a significant increase is also observed in blood pressure with 393 TT genotypes (FIG. 26C), which does not occur with CC (FIG. 26A) and TC genotypes (FIG. 26B).

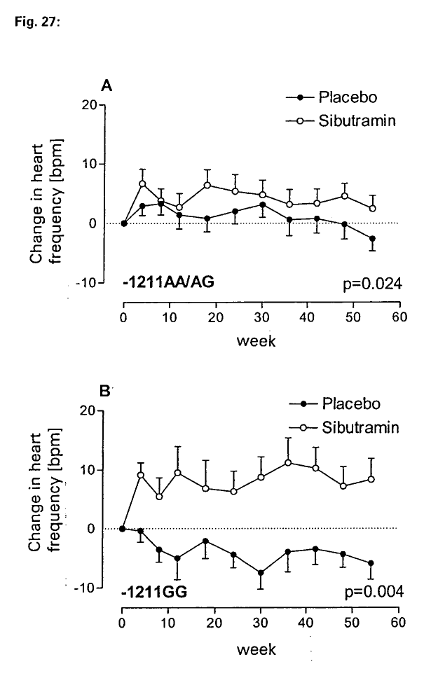

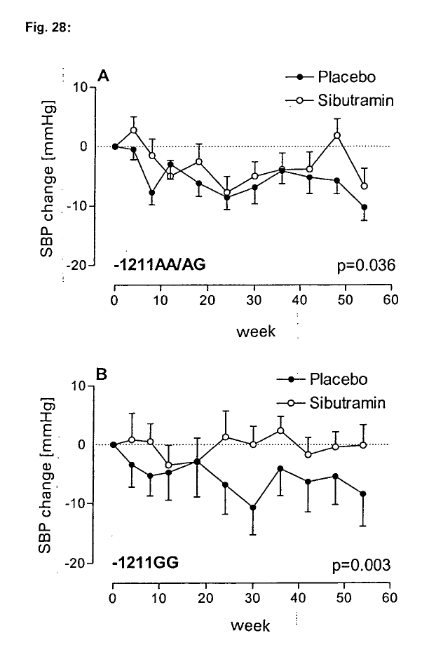

FIG. 27 illustrates once again the possible use of a gene modification in the human GNAS gene to predict an increase in the heart rate during therapy with sibutramine. This illustrates the effects, in dependence on genotypes of the G(-1211)A polymorphism. With respect to the G(-1211)A polymorphism, with GG genotypes (FIG. 27B) there is a significantly greater change in heart rate (sibutramine versus placebo) than with AA/AG genotypes (FIG. 27C). Finally, FIG. 28 illustrates the possibility of an additional use of a gene modification in the human GNAS gene in the prediction of an increase in systolic blood pressure during therapy with sibutramine. The illustration shows the changes in time of the systolic blood pressure (SBP), in dependence on the genotypes of the G(-1211)A polymorphism. Under therapy with sibutramine there is then a lack of reduction of the systolic blood pressure with -1211 GG genotypes (FIG. 28B). The difference in the blood pressure change is significantly higher than with AA/AG genotypes (FIG. 28A). These observations are of great therapeutic importance as it is particularly patients with GG genotypes who lose enough weight with low calorie food and changes in life style, so that they do not benefit from therapy with sibutramine.

The use of gene modifications is therefore a suitable method of identifying patients who develop cardiovascular side-effects during treatment with drugs. These effects can be caused directly, in that these drugs stimulate receptors which evoke either vasoconstriction or an increase in heart rate mediated through Gαs. These include, for example, sibutramine, triptans and noradrenaline/serotonin re-uptake inhibitors. This also includes drugs which inhibit the breakdown of catecholamines (MAO inhibitors) and tricyclic antidepressives. This also includes drugs which cause a reduction in blood pressure and induce activation of the sympathetic nervous system by reflex action (e.g. sildenafil and other inhibitors of phosphodiesterases, nitrates).

A further proof for the general application of gene modifications in the GNAS gene to predict the actions of drugs results from the observed genotype dependence of the clinical course of bladder carcinoma (FIG. 15), renal cell carcinoma (FIG. 16) and acute myeloid leukemia (FIG. 17). These patients were all treated with different drugs. Use of gene modifications in the GNAS gene shows different clinical courses and this demonstrates different responses to these forms of therapy.

Classification of the Invention

Polymorphisms in the GNAS gene are described which are in mutual coupling disequilibrium and which, either alone or in any combination, can be used to predict the clinical course of disease or the actions of drugs. The first part of the invention consists of the detection of gene modifications and the detection of haplotypes or coupling disequilibria. The second part of the invention describes the use of gene modifications to predict risks of disease and the clinical course of diseases. The basis for this is the altered activability of Gαs on the basis of the described gene modifications. These then also determine the gene status-dependent different response to drugs. This raises the question of whether the simultaneous use of gene modifications for the described purposes is unexpected, or whether this is known to the expert. This will be demonstrated on the basis of an example.

This close relationship has already been described for the GNB3 gene. With these gene modifications too, which lead to a change in the function of a Gβ₃-subunit, risks of disease, clinical courses of disease and the response to different drugs can be predicted. A C825T polymorphism in gene GNB3 is in coupling disequilibrium with other polymorphisms in the same gene, so that specific haplotypes can be described (D. Rosskopf et al., Identification and ethnic distribution of major haplotypes in the gene GNB3 encoding the G protein beta3 subunit. Pharmacogenetics 12 (3):209-220, 2002). These gene modifications are associated with increased risks for quite different diseases, e.g. hypertension, obesity, stroke, myocardial infarction, insulin resistance, diabetes and depression (P. Zill et al., Evidence for an association between a G protein beta₃-gene variant with depression and response to antidepressant treatment. Neuroreport 11 (9):1893-1897, 2000.; W. Siffert et al., G protein b₃ subunit 825T allele and its potential association with obesity in hypertensive subjects. J. Hypertens. 17:1095-1098, 1999.; W. Siffert et al. Association of a human G protein beta₃ subunit variant with hypertension. Nat. Genet. 18 (1):45-48, 1998; R. A. Hegele et al., G protein beta₃ Subunit Gene Splice Variant and Body Fat Distribution in Nunavut Inuit. Genome Res. 9 (10):972-977, 1999. C. K. Naber, et al., Interaction of the ACE D Allele and the GNB3 825T Allele in Myocardial Infarction. Hypertension 36 (6):986-989, 2000; A. C. Morrison et al., G protein beta₃ Subunit and alpha-Adducin Polymorphisms and Risk of Subclinical and Clinical Stroke. Stroke 32 (4):822-829, 2001. T. C. Wascher et al. Associations of a human G protein beta₃ subunit dimorphism with insulin resistance and carotid atherosclerosis. Stroke 34 (3):605-609, 2003. D. Rosskopf et al. Interaction of the G Protein beta₃ Subunit T825 Allele and the IRS-1 Arg972 Variant in Type 2 Diabetes. Eur. J. Med. Res. 5 (11):484-490, 2000.)

These gene modifications in the GNB3 gene are also associated with response to quite different drugs. These also include drugs of which the activity is not directly mediated through G-proteins. Examples include: hydrochlorothiazide (activity not directly mediated through G-protein), sildenafil (activity not directly mediated through G-protein), vaccination against hepatitis B (activity not directly mediated through G-protein), a variety of antidepressives (activity not directly mediated through G-protein), sibutramine (activity not directly mediated through G-protein), antidiabetics (activity not directly mediated through G-protein) and drugs to treat leukemia (activity not directly mediated through G-protein). In addition and as expected, there is prediction of the action of drugs which activate or inhibit G-protein coupled receptors, e.g. of clonidine, propanolol, noradrenaline, endothelin, angiotensin etc. (J. M. Fernandez-Real et al, G Protein beta₃ Gene Variant, Vascular Function, and Insulin Sensitivity in Type 2 Diabetes. Hypertension 41 (1):124-129, 2003; P. Zill et al; Evidence for an association between a G protein beta₃-gene variant with depression and response to antidepressant treatment. Neuroreport 11 (9):1893-1897, 2000. R. R. Wenzel et al., Enhanced vasoconstriction to endothelin-1, angiotensin II and noradrenaline in carriers of the GNB3 825T allele in the skin microcirculation. Pharmacogenetics 12 (6):489-495, 2002. S. T. Turner et al., C825T Polymorphism of the G Protein beta(3)-Subunit and Antihypertensive Response to a Thiazide Diuretic. Hypertension 37 (2 Part 2):739-743, 2001. R. F. Schäfers et al., Haemodynamic characterization of young normotensive men carrying the 825T-allele of the G protein beta₃ subunit. Pharmacogenetics 11 (6):461-470, 2001. M. Ryden et al., Effect of the (C825T) Gbeta(3) Polymorphism on Adrenoceptor-Mediated Lipolysis in Human Fat Cells. Diabetes 51 (5):1601-1608, 2002. H. Hauner et al., Prediction of successful weight reduction under sibutramine therapy through genotyping of the G protein beta₃ subunit gene (GNB3) C825T polymorphism. Pharmacogenetics 13 (8):453-459, 2003. OH. J. Lee et al., Association between a G protein beta₃ subunit gene polymorphism and the symptomatology and treatment responses of major depressive disorders. Pharmacogenomics. J., 2003. A. Mitchell et al., Venous response to nitroglycerin is enhanced in young, healthy carriers of the 825T allele of the G protein beta₃ subunit gene (GNB3). Clin. Pharmacol. Ther. 74 (5):499-504, 2003. H. Nuckel et al., The CC genotype of the C825T polymorphism of the G protein beta₃ gene (GNB3) is associated with a high relapse rate in patients with chronic lymphocytic leukaemia. Leukemia & Lymphoma 44 (10):1739-1743, 2003. J. Nürnberger et al., Effect of the C825T polymorphism of the G protein beta₃ subunit on the systolic blood pressure-lowering effect of clonidine in young, healthy male subjects. Clin. Pharmacol. Ther. 74 (1):53-60, 2003. A. Serretti et al., SSRIs antidepressant activity is influenced by Gbeta₃ variants. Eur. Neuropsychopharmacol. 13 (2):117-122, 2003. H. Sperling et al., Sildenafil Response is Influenced by the G Protein beta₃ Subunit Gnb3 C825t Polymorphism: A Pilot Study. J. Urol. 169 (3):1048-1051, 2003. M. Lindemann et al., Role of G protein beta3 subunit C825T and HLA class II polymorphisms in the immune response after HBV vaccination. Virology 297 (2):245-252, 2002)

It should thus be assumed that the presently described connections between the described gene modifications in the human GNAS gene and the prediction of disease risks, clinical courses and the response to disease therapies by pharmacological and non-pharmacological means and the prediction of undesired drug reactions (side-effects) will also be present to the extent that they have not been proven by scientific investigation.

1-9. (canceled)

10. A process to predict disease risks and/or clinical courses of disease and/or drug actions, drug side-effects, reaction on pharmacological and non-pharmacological therapeutic measures and/or the identification of modifications in gene expression in different diseases and/or drug targets which are associated with gene modification in the human GNAS gene, in which a gene modification in the gene for human G protein Gαs-subunit is identified, comprising the determination of a base change (polymorphism) in the promotor region and/or in intron 1 of the gene and wherein the base change in the promotor region is selected from the G(-1211)A and/or G(-839)T and wherein the base change in intron 1 is selected from one or more of D1340I, T1368C, A2025G, C2273T, T2291C and C2445G.

11. The process according to claim 10, wherein, in addition, the T393C polymorphism is identified.

12. The process according to claim 10, wherein a combination of these polymorphisms is investigated.

13. The process according to claim 10, wherein the optimal dosage of a drug or the duration of therapy for a patient is predicted.

14. The process according to claim 10, wherein a gene change is identified which is in a coupling disequilibrium with one of the polymorphisms of claim 1.

15. A gene test kit comprising means for the identification of a base change (polymorphism) in the promotor region and/or in intron 1 of the human GNAS gene which codes for the Gαs-subunit in the human G protein and wherein the base change in the promotor region is selected from G(-1211)A and/or G(-839)T and wherein the base change in intron 1 is selected from one or more of D1340I, T1368C, A2025G, C2273T, T2291C, and C2445G.

16. The gene test kit according to claim 15, wherein the determination of the base change is performed by direct sequencing, restriction analysis, reverse hybridisation, dot-blot- or slot-blot processes or according to the multiplex-PCR and by hybridisation on a DNA-chip.
